(19) United States
(12) Patent Application Publication
(10) Pub. No.: US 2008/0187954 A1
(43) Pub. Date: Aug. 7, 2008

(54) Method For Producing Antibodies

(75) Inventors: Robert Kallmeier, Buckinghamshire (GB); Gilles Reiss, Basel (CH); Robert Gay, North Bondi (AU); Stephan Kalwy, Berkshire (GB)

Correspondence Address: WENDEROTH, LIND & PONACK, L.L.P., 2033 K STREET N. W., SUITE 800, WASHINGTON, DC, 20006-1021, US

(73) Assignees: Lonza Ltd., 4002 Basel (CH); Lonza Biologics PLC, Berkshire (GB)

(21) Appl. No.: 10/591,812

(22) PCT Filed: Mar. 10, 2005
(86) PCT No.: PCT/EP05/02538

§ 371 Date: Sep. 26, 2006

Related U.S. Application Data
(60) Provisional application No. 60/551,353, filed on Mar. 10, 2004.
(60) Provisional application No. 60/554,914, filed on Mar. 22, 2004.
(60) Provisional application No. 60/600,775, filed on Aug. 12, 2004.

(51) Int. Cl. C12P 21/06

(52) U.S. Cl. 435/68.1

(57) ABSTRACT

A novel method for expression of immunoglobulines is devised.

The present invention relates to the field of biopharmaceuticals, in particular to a method of producing recombinant antibodies.

Monoclonal antibodies are becoming an ever more important class of pharmaceuticals. The elevated cost of manufacture entails a particular need to improve the primary yield of antibody in the cell culture supernatant to the utmost extend possible. Yield enhancement at the transcriptional level has been widely optimized, using strongest possible promoters and enhancer elements of mostly viral origin. Alike, as regards cell culture methodology and cell culture media, huge progress has been made for achieving high density growth, high specific productivity and prolonged viability of cultured cells.

Therapeutic antibodies are mostly of the IgG-type, them being 150 KDa tetramers made up from two different sets of protein chains, a heavy (50 KDa) and a light chain (25 Kda). All chains made of multiple domains of the Ig-domain fold class. In particular the heavy chains are not only glycosylated but the proper use of glycosylation sites and the correct composition of the carbohydrate moiety strongly affect quaternary structure. For instance, it has been found a decade ago that certain deposits of aggregated antibody correlated with a terminal galactosylation defect of the carbohydrate moiety of aggregated antibody. Further, there are intra- and interchain disulfide bonds contributing to assembly and stabilizing the antibody structure under adverse extracellular conditions. In short, folding and assembly are equally crucial to efficient expression of antibody in recombinant cells.

Unfortunately, despite refined genetic expression technology, often a considerable portion of the light and heavy chains are not properly assembled. Partly, it is very difficult to balance optimally light and heavy chain expression by promoter activity without having the one or other chain in excess. Further, there are assembly problems of the monomeric protein chains; even in the presence of 70% free IgG light chain, considerable amounts of monomeric IgG heavy chain may be detectable. Excess light chain may also be shedded to the cell culture medium along with functional, fully assembled monoclonal antibody (Mab). The simple chemical law of mass action does not apply in a cell. Efforts to elevate selectively the expression level of the heavy chain have not proven successful. Gass et al., Trends Immunol. 15/1 (2004) suggest that in plasma cells, that is actively antibody secreting B-cells, the best achievable Mab titre is reached in the presence of excess light chain. Likely explanations are inefficient assembly and/or premature selective degradation targeting of the heavy chain; quality control and degradations timing are known to involve glucose tagging of the carbohydrate moieties. Protein domain assembly may be driven by affinity interactions, formation of disulfide bridges bringing domains into close proximity and/or the need to bury exposed more hydrophobic patches on the surface of individual domains; in the initial stage of assembly and folding, it is believed that such less patches are shielded by chaperone proteins. It is well-known nowadays that secretory glycoprotein is folded and assembled to higher order complexes in the inner, endoplasmic compartment (ER) of the cell structure only shortly after protein synthesis. The ER is the sole compartment to comprise specific auxiliary assembly factors along with quality control mechanism (Ellgaard et al., Quality control in the secretory pathway, Science. 1999 Dec. 3; 286:1882-8; Helenius et al., Intracellular functions of N-linked glycans, Science. 2001 Mar. 23; 291:2364-9.). Once the chance for successful assembly has passed, no assembly will further take place along further passage through the secretory pathway of the cell. In fact, some cell types such as CHO cells secrete unassembled chains of both type whereas others such as NSO cells retain selectively non-assembled chains of the IgG heavy type only and probably target them to degradative pathways.

Davis et al. Effect of PDI overexpression on recombinant protein secretion in CHO cells, 2000, Biotechnol. Prog. 16:736-743) seeked to increase secretion rate of a recombinant product protein comprising disulfide bridges in a widely employed industrial host cell system, namely CHO cells. Protein disulfide isomerase (PDI) was co-expressed with the product protein in addition to the endogenous expression level of PDI. Contrary to exceptions, co-expression of PDI along with product protein led to reduced secretion rate and thus productivity; further, the product protein was retained inside the cells in the endoplasmic reticulum, colocalizing with PDI enzyme protein in the endoplasmic reticulum.

WO03/057897 teaches a method for expressing a recombinant protein comprising co-expression of chaperone proteins and small heat shock proteins. Those additional proteins are said to promote successful folding and assembly and thus the portion of correctly folded, most active product protein.

As a disadvantage, the co-expression of several auxiliary factors may decrease total expression rate of product protein and requires careful optimization of individual co-expression rates of such auxiliary factors. Different product protein might dependent to a varying degree on individual, only partially overlapping chaperone functions of which a multitude has become known to date, e.g. GroEL, GroES, DnaK, DnaJ, GrpE, ClpB, IbpA, IbpB . . . . This whilst it is not desirable to co-express all of them at a time at the sole expense of product protein production rate.

Shu et al. (Proc. Natl. Acad. Science USA, 1993 September, 90: 7995-7999) describe construction of a single-chain immunoglobulin-like molecule that comprises the hinge and Fc portion (CH2+CH3) of an IgG-type antibody, in contrast to the more routinely employed single chain antibodies made up from isolated variable domains. The design of the single-chain monomeric protein included a covalent linkage of the carboxyl terminus of the VH domain and the amino end of the VL domain through a (Gly-Gly-Gly-Gly-Ser)3 peptide linker. The carboxyl end of VL and the amino end of CH2 were joined through the hinge region. The single chain assembled into a dimeric molecule.

The idea behind this approach was that therapeutic effect will require the effector functions of whole antibodies having Fc constant portions. Furthermore, it is technically less demanding to express such whole fused antibody from a single host cell than by efficiently introducing and co-expressing two separate genes from heavy and light chain separately Smaller fusion molecules such as commonly employed scFV antibody fragments suffer both from lack of effector function as well as from a lower affinity of antigen binding, despite identical variable portions. Further, their plasma half life is much shorter—whole antibodies having Fc portions are much less rapidly cleared from a patient's body.

The disadvantage behind any such single fusion whole antibody gene approach is, however, that the longer the residential time of such antibody in a patient's body, the more relevant immunogenicity of any non-natural, extended portion of such artificial fusion protein becomes. The linker peptide represents such potentially immunogenic portion. It will likely be the more immunogenic the longer it is; the composition of the linker peptide sequence may further influence its immunogenicity. Of course, immunogenicity of the fusion protein and especially the linker portion of the protein may be diminished by further covalent modification such as PEGylation of the antibody/the linker portion with polyethyleneglycol chains; such modification requires additional downstream processing of product protein and expensive clinical grade chemical reagents though. One problem remains though, namely that the linker, attached to the N-terminus of the VL chain tends to obstruct access to the antigen binding site or complementarity determining region (CDR) cavity by steric hindrance and/or may affect proper pairing of the VL and VH domains to constitute the proper antigen binding site by pairing their two CDRs. The extent of such problem may differ from antibody to antibody, depending on the antibodies' specificity/set of CDRs from both variable domains and chemical properties/size of the antigen sought to be detected/bound. The aspect of steric hindrance will become the more problematic if PEGylation of the linker portion is to be considered, for above said reasons. Further, given the purposeful conformation flexibility of the linker, the linker may by virtue of the proximity effect also show reversible, low affinity binding to at least some antigen binding sites like a competitive inhibitor, affecting antibody binding even for small antigens. In short, the approach of Shu has several severe shortcomings that prevent it from being broadly applicable to IgG-type antibodies in general.

An approach analogous to Shu, but with duplicated variable domains and an accordingly longer peptide linker is described in Santos et al. (1999, Clinical Cancer Research 5:3118-3123). The same disadvantages brought forward in relation to Shu et al. apply to this analogous approach, of course.

It is the object of the present invention to avoid the disadvantages of the prior art and to devise another method of expression of standard, tetrameric whole IgG antibody having Fc receptor activity and consisting of at least two different polypeptide chains. This object is solved by a method for producing an immunoglobuline having Fc receptor activity or complement activation activity which immunoglobuline when secreted from a vertebrate host cell comprises at least a first and a second polypeptide chain which two polypeptide chains are different, comprising the steps of
a. expressing in a vertebrate host cell having Golgi-only or late-Golgi-only resident furin family endoprotease activity an fusion polypeptide comprising a secretion targeting sequence directing the polypeptide to the secretory pathway and further comprising at least the first and second polypeptide sequences and at least one cleavage site for the said endoprotease activity
b. having the fusion polypeptide cleaved in the cells by the furin family endoprotease activity into the first and second polypeptide chains and
c. harvesting the secreted immunoglobuline.

First and foremost, the method of the present inventions allows of removing partially or completely linker portions from the N-terminus of a variable domain, be it a VH or a VL domain, hence to avoid the above said inhibitory effects of (elongated) linker segments on the binding of an antigen to an antibody that is specific for said antigen. Furthermore, the invention allows surprisingly of improved expression levels of the fused and subsequently endoproteolytically cleaved, fully functional antibody secreted into the medium as compared to conventional, separate two chain heterodimeric expression of antibody in a given host cells. The extent to which expression levels are improved is highly cell line dependent. In some cell lines, enhancement was moderate but significant whilst in others, the enhancement was surprisingly drastic for a given IgG antibody.

An immunoglobuline according to the present invention has Fc-receptor activity or complement activation activity or both. Whereas complement activation is clearly defined in the art as to relate to induction of blood clotting (by possibly different pathways), Fc-receptor activity in the context of the present invention is to be understood as to the activation of cellular Fc receptors which trigger a cellular response, e.g. in the case of naturally occurring IgG or IgA triggered phagocytic or cytotoxic activities or e.g. in the case of release of mast cell granula upon triggering of cellular receptors by natural IgE class immunoglobulin. Similarly, amongst natural antibodies, both IgM and IgG class antibodies may trigger complement activation. It goes without saying that any such effector activities may vary amongst naturally occurring subclass antibodies and their known allotypes, and accordingly may vary amongst the antibodies of the present invention. In the context of the present invention, however, it is possible that Fc-receptor activity or complement activation effector domains are engineered into any given immunoglobulin structure by means of domain swapping, effectively transferring or adding the respective effector properties in such resulting immunoglobulin. The immunoglobulin may be a naturally occurring type of immunoglobuline, apart from its specific binding for a given antigen, or it may be an engineered, artificial type of immunoglobuline. This includes species-chimeric antibodies or CDR grafted antibodies, antibodies created by gene shuffling or site-directed engineering, antibodies chemically modified with PEG or radioisotope-chelating moieties or fusion proteins linking an immunoglobulin moiety having afore said activity to any other proteinaceous moiety such as another enzymatically active domain. The extend to which every activity is conferred by a given Immunoglobuline may vary. Both types of effector function are caused by the constant portion regions of the immunoglobuline heavy chain; for instance, the different human IgG subclasses vary in their relative efficacy to activate and amplify the steps of the complement cascade. In general, human IgG1 and IgG3 most effectively fix complement, IgG2 is less effective, and IgG4 does not activate complement. Assay formats to test for either of afore said activities are well-known to immunologists and other persons; suitable protocols may e.g. be found in standard immunochemistry lab manuals such as Harlow et al., Antibodies—a laboratory manual, Cold Spring Harbor Laboratory Press 1988. In naturally occurring immunoglobuline for instance, light chains have a single constant region domain and heavy chains have several constant region domains.—All human subclasses IgG1, IgG2, IgG3 and IgG4 mediate cytotoxic effector functions through constant chain portions (ADCC: antibody directed cytotoxicity), brought about by interaction of the antibody with killer cells/cytotoxic T-lymphocytes; this is quite notable because IgG4 has often been said not to mediate such effects. However, it has been found that consistently, human IgG4 is intrinsically capable of mediating ADCC whilst its extent is strongly modulated/dependent on the source of effector cells used in the assays such as ⁵¹Cr-release, due to a distinct natural polymorphism in humans at least. This has been shown by Greenwood J, Clark M, Waldmann H.: Structural motifs involved in human IgG antibody effector functions Eur J Immunol 1993; 5: 1098-1104.

The naturally occurring antibody or immunoglobulin classes IgG and IgA naturally have three constant region domains, designated CH1, CH2 and CH3, and the IgM and IgE classes have four constant region domains. In contrast, e.g. WO02/056910 devises artificial antibodies for human therapy that are devoid of the CH1 domain; such antibodies are encompassed by the notion of immunoglobulin according to the present invention as well.

In the context of the present invention, secretion is understood in the usual way as to mean release of matter from the outer cellular membrane to the surrounding, extracellular space. The method according to the present invention does not only allow

A host cell according to the present invention may be any vertebrate host cell line that can be, in contrast to primary cell lines, stably propagated in cell culture. Possible cell lines are e.g. COS cells, NSO cells, CHO cells, HT1080 cells, PER-C6 cells, BHK cells, Sf-9 cells, 293 or 293-EBNA cells. In certain further possible embodiments of the present invention, it may also be possible to use plant cells including algal cells that may be grown in suspension cell culture which plant cells allow of having such antibody secreted from said plant cell under circumstances.

Preferably, the vertebrate host cells according to the present invention are mammalian cells, most preferably human cells such as e.g. HT1080 cells, 293, 293-EBNA or HBK-11 cells (ATCC-CRL12569; also see U.S. Pat. No. 6,136,599). More preferably, the human cells according to the present invention are selected from the group consisting of HT1080 cells and Per-C6 cells (Crucell B. V., Netherlands; WO97/00326, also see EP-1161548). Most preferably, the cells are HT1080 cells. For instance, HT1080 cells can be ordered as ATCC No. CCL-121 at the Amercian Type Culture Collection, Manassas/VA, U.S.A. HT1080 have been found to allow of enhanced product glycosylation when used in combination with glutamine synthetase selection marker system (WO 03/064630).

Further preferred is that the cells are CHO cells, more preferably CHO-K1 cells and most preferably CHO cells adapted for growth in serum-free suspension culture (i.e. excluding microcarrier-borne culture). Suitable media for serum-free suspension culture of CHO cells are commercially available (e.g. CD-CHO from Invitrogen Inc.).

Preferably, the host cells as specified above are lymphoid cells, more preferably mammalian lymphoid cells, encompassing e.g. hybridoma, myeloma and trioma cells lines. Examples are e.g. non-secreting hybridoma such as SP2/0 and non-secreting myeloma cells e.g. such as NSO cell line ECACC No. 85110503 (European Collection of Cell cultures, Centre for Applied microbiology, Salisbury/Wiltshire SP4 0JG, United Kingdom) from mouse or YB2/3.0 Ag20 (described in GB2070313) from rat. Myeloma cells such as NSO cells truly are B-lymphoid cell types, namely plasmacytoma cell lines, although being routinely addressed in the art as ‘myelomas’ (Barnes et al., Cytotechnology 32:109-123, 2000). More preferably, the lymphoid vertebrate host cells are mammalian lymphoid cells, most preferably they are non-secreting rodent myeloma cells. As said below for the specific example of lymphoma proprotein convertase, such cell lines comprise suitable levels of endogenous furin endoprotease activity. Lymphoid cell lines are particularly preferred in combination with the preferred embodiment of furin endoprotease activity according to the present invention consisting only of an endogenous enzyme activity of the host cell.

It goes without saying that the level of furin endoprotease activity expressed in any of the above specified host cell types determines the extent to which the immunoglobuline molecule as finally secreted into the cell culture medium according to the present invention has indeed been cleaved by the endoprotease activity. Thus, a mixture of secreted immunoglobuline made up from uncleaved, fused and cleaved immunoglobuline polypeptides may be obtained. From such mixture, the fraction of cleaved, assembled immunoglobuline polypeptides can be obtained by further chromatographic separation techniques that are routine in the art. It might also be possible to remove uncleaved polypeptide by means of an affinity chromatography with an e.g. antibody-based stationary phase specifically recognizing and binding to the linker peptide.

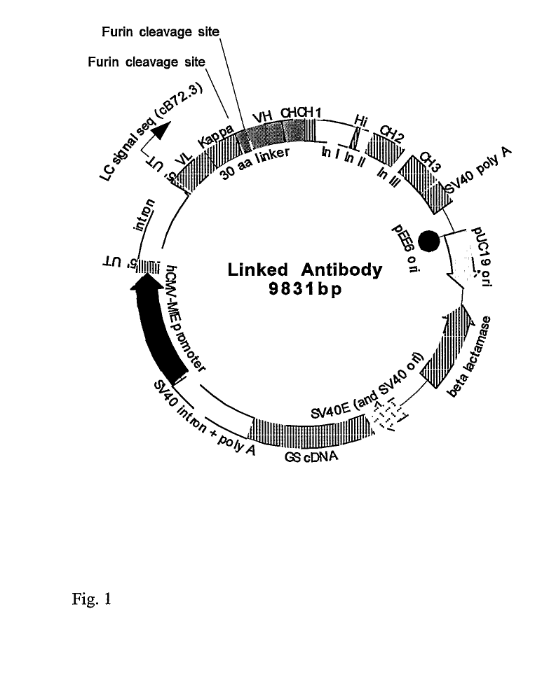

Preferably, the immunoglobuline or Ig molecule comprises at least a hinge domain, a CH₂ and a CH3 domain or functional variants thereof. Those domains form the essential Fc part e.g. in natural IgG. Detailed descriptions and definitions of these structural elements of an immunoglobuline are set forth in Amzel et al., Three-dimensional structure of immunoglobulins, Ann. Rev. Biochem. 48, 961-997 (1979); Davies et al., Structural basis of antibody function, Ann. Rev. Immunol. 1, 87-117 (1983); Hunkapiller et al., Diversity of immunoglobuline gene superfamily, Adv. Immunol. 44, 1-63 (1989). Said domains can be naturally occurring domains, artificially created chimeric versions of such domains or chimeric assemblies of such domains or versions engineered e.g. by site-directed mutagenesis. In the past, chimeric, CDR grafted mouse human chimeric antibodies were often used; alike, potential glycosylation sites in the variable or CH1/CL domain portions were often eliminated by site directed mutagenesis. Of course, the extend of engineering of any part of the immunoglobuline according to the present invention may be often limited by the need to avoid creating extended, strongly immunogenic motifs in engineered antibody, apart from the natural variability inherent to the complementarity determining regions. Apart from this, for the antigen-binding moiety upstream of the hinge portion that is conventionally coined the Fv portion of e.g. IgG type antibody, the only requirement according to the present invention is that such portion is made up from two distinct polypeptide chains (when secreted) and has some antigen-binding property. It is possible that an immunoglublin according to the present invention has increased antigen-binding valency achieved by multiplied variable domains arranged in a perl-on-a-string fashion in its ‘Fv’ portion (similar to the suggestive drawing in FIG. 1 of Santos et al., Clinical Cancer Research, Vol. 5, 3118-3123, October 1999, though the very antibody devised in Santos et al. is based on the scFv concept employing very short interdomain linkers and thus probable has a different domain pairing pattern that shown in FIG. 1, making the large linker peptide shown in FIG. 1 a mandatory feature of Santos' antibody, in contrast to the present invention). Such ‘Fv’ portion, or what can be considered the equivalent of a naturally occurring Fv portion, may also be e.g. a shortened version that is devoid of the CH1 and CL domain, or e.g. replace or enhances the CH1/CL domain paring by a variable number of any other given, interfacing domain pair (e.g. VL/H domains or pairing domains unrelated to immunoglobuline but stemming from man, such as not being immunogenic). Essentially, an immunoglublin according to the present invention is an antibody allowing of triggering Fc-receptor and/or complement activation activity and further comprising said domain elements; a functional variant of a known, natural domain equally complies with this requirement. The latter two activities appear both to located on or near the CH2 domain, but likely constitute different epitopes and are influenced by neighbouring domain elements and the tertiary and quaternary structure of the immunoglobulin. Proper domain interfacing as well as the feature of structurally important N-glycosylation and respective N-glycan structure at Asn-297 (Roy Jefferis, Glycosylation of human IgG antibodies, Biopharm 2001, Advancstar Publication/U.S.A.; numbering from natural human IgG), spacing CH2 domains properly apart, may further influence said activities (Roy Jefferis, ibd.; Lund et al. Multiple Interactions of IgG with its core oligosaccharide can modulate recognition by complement and human FcγRI and influence the synthesis of its oligosaccharide chains, J. Immunol. 164, 4178-4184 (2000)) Complement activation is initiated by binding of C1q, a subunit of the first component C1 in the blood clotting cascade, to an antigen-antibody complex. Even though the binding site for C1q is located in the CH2 domain of a natural antibody, the hinge region influences the ability of the antibody to activate the cascade—recombinant immunoglobulins lacking a hinge region are unable to activate complement. Studies have indicated that the hinge length and segmental flexibility correlate with complement activation; however, the correlation is not absolute. Human IgG3 with altered hinge regions that are as rigid as IgG4 still effectively activate the cascade. The hinge region is naturally found in IgG, IgA and IgD classes; as said already, it acts as a flexible spacer, allowing the Fab portion to move freely in space. In contrast to the constant regions, the naturally occurring hinge domains are structurally diverse, varying both in sequence and length amongst Immunoglobuline classes and subclasses. For example, three human IgG subclasses (IgG1, IgG2, IgG4) have hinge regions of 12-15 amino acids whilst the fourth, IgG3, comprises approximately 62 amino acids, including 21 proline residues and 11 cysteine residues. Crystallographic studies allowed of dividing the hinge region functionally into three different subregions: upper, core and lower hinge (Shin et al., Immunological Rev. 130:87 (1992)). The upper hinge includes amino acids from the carboxyl end of CH1 to the first residue in the hinge that restricts motion, generally the first cysteine residue that forms an interchain disulfide bond between the two heavy chains. The length of the upper hinge region correlates with the segmental flexibility of the antibody. The core hinge region contains the inter-heavy chain disulfide bridges, and the lower hinge region joins the amino terminal end of the CH2 domain and includes residues in CH2 (Shin et al., supra). The core hinge region of human IgG1 contains the sequence Cys-Pro-Pro-Cys which after formation of disulfide bonds results in a cyclic octa-peptide structure which may act as a pivot conferring flexibility. The hinge region may also comprise carbohydrate attachment sites, e.g. human IgA1 contains five carbohydrate sites within a 17 amino acid segment of the hinge region, conferring exceptional protease resistance to the hinge region.

Lack of the hinge region also affects the ability of human IgG immunoglobulins to bind Fc receptors on immune effector cells. Binding of an immunoglobulin to an Fc receptor facilitates antibody-dependent cellular cytotoxicity (ADCC), which is presumed to be an important means to eliminate tumour cells. In the context of the present invention, Fc receptor activity is understood as ADCC activity as can be assayed with an appropriate target cell expressing the appropriate antigen by standard ⁵¹Cr-release assay (see. e.g. Harlow et al., supra) or any more modern methods (e.g. Patel et al., J. Immunol. Methods. 1995 Jul. 17; 184(1): 29-38). In the context of the present invention, it is sufficient to find immunological effector cells (blood cells) from at least one species working in such assay as compared to an inactive, unrelated protein standard (e.g. serum albumin). Preferably, the immunological effector cells used to kill the target cells by means of ADCC in the assay are from human.—The human IgG Fc receptor family is divided into three groups Fcγ RI (CD64) which is capable of high affinity binding of human IgG, and Fcγ RII (CD 32) and Fcγ RIII (CD16), both of which are low affinity receptors. The molecular interaction between each of the three receptors and immunoglobulin has not been defined precisely but experiments indicate that residues in the hinge proximal region of the CH2 domain are important to the specificity of the interaction between the antibody and the Fc receptor (Lund et al., ibd.; Shields et al., 2001, J. Biol. Chem. 276:6591-6604). In addition, IgG1 myeloma protein and recombinant IgG3 chimeric antibodies that lack a hinge region are unable to bind FcγRI, purportedly because accessibility to CH2 is decreased (Shin et. al., Intern. Rev. Immunol. 10:177, 178-179 81993).

Preferably, an immunoglobulin according to the present invention is of the IgG structural type and that the first polypeptide is an Ig-Light chain (L) comprising one VL and a CL domain, and that the second polypeptide is an Ig-Heavy Chain (H) comprising one VH, a CH1, a CH2 and a CH3 domain and a hinge domain. In this context, V stands for Variable domain comprising the complementarity determining region that forms the antigen binding pocket. H stands for heavy chain, L stands of course for light chain, C for Constant domain. More preferably, the CH1 to CH3 and hinge domain are of human IgG class or subclass or allotype.

Preferably, alone or in combination with any other preferred embodiment cited, the fusion polypeptide according to the present invention comprises the sequences of the first and second polypeptide separated by a linker. More preferably, the linker is positioned such as that the Light and Heavy Chain are separated by a linker and that the linker is cleaved off from both Heavy and Light Chain by the furin family endoprotease activity. The linker according to the present invention is a linker peptide of course, linking heavy and light chain at the level of translation by allowing translation as a common fusion polypeptide from a single open reading frame. Examples of a suitable linker peptides linking heavy and light chain, or what can be said to correspond to them, can be found e.g. in Santos et al., ibd., or Shu et al. In general, a linker peptide should mainly comprise amino acids that promote an extended, fully solubilized conformation; small or possibly hydroxylated side chains such as found in glycin or serine would be first choice. In a preferred embodiment, the linker comprises one or several oligomers consisting of afore said amino acids glycine and serine. Equally preferred, alone or in combination, is that the linker is characterized by a content of >60% of all residues being glycine. Examples of such are (GGGGS)₆ or [AG₃S(G₄S)₂]₂. For a natural IgG-type immunoglobuline, preferably a linker's minimal length should be about 24-40 amino acids spanning the Light Chain's C-terminus to the Heavy Chain's N-terminus for example. Preferably, the linker comprises at least 20 amino acids. It goes without saying that any engineering by addition or deletion of domains of such natural antibody type would affect the minimal linker length required.

A particularly preferred embodiment according to the present invention is that the linker as defined above and further below with regard to its further features, spans from the C-terminus of an immunoglobuline light chain not harboring any effector functions such as ADCC and/or complement activation to the N-terminus of an immunoglobuline heavy chain harboring said effector functions. It has been found that in such configuration, the linker portion can be efficiently cleaved off at or within the linker, proximal to the linker-heavy chain junction. This way, in minimum only one cleavage site near or at the N-Terminus of the Heavy chain is required; the basic residues of the cleavage signal will remain on the linker's C-terminus after cleavage, avoiding any environmental modifications near or at the antigen binding site of the VH domain. Optionally, the linker may remain as an appendix on the light chain portion then, having no adverse effect on effector functions of the heavy chain. Further, independent of the number of cleavage sites applied in the linker, such approach avoids of adding basic residues to the C-terminus of the heavy chain where they could deteriorate the biological half-life of antibody. More preferably, where the linker is spanning in N to C-terminal direction from Light to Heavy chain, at least one cleavage site is positioned at or within the range of residues -15 to -1 of the linker-heavy chain junction.

The furin endoprotease activity is an endoprotease activity naturally located exclusively in the late Golgi compartment or secretory vesicles further downstream in the secretory pathway. Usually, nascent newly synthesized furin-family endoproteases are proteolytically activated upon exit from the ER compartment only and gain full enzyme activity due to the distinctive pH and ionic strength (including particularly Ca2+ levels) features of the late Golgi or of the dense secretory vesicles in case of regulated secretion, as e.g. with proinsulin. Furin family endoproteases are Ca²⁺ dependent in general. The furin endoprotease family is a family of mammalian endoproteases sharing a common motif of catalytical residues forming the active site and further a domain motif termed the P or Homo B domain. Examples from mammalian cells are e.g. furin (PACE), the pro-insulin convertases PC2 and PC3 or the furin homologue PACE 4. In baker's yeast, the mating or alpha-factor processing enzyme kex 2 is a homologue of mammalian furin endoproteases. A detailed description of said features and further references describing said two domain motif features are provided in the review Steiner et al., 1996, The role of prohormone convertases in insulin biosynthesis, Diabetes & Metabolism 22:94-104. The proteases of the furin endoprotease family cleave selectively at di- or multibasic sites in the primary sequence. Most prohormones and neuroendocrine precursors, including proinsulin and proglucagon, that are processed and stored in dense core secretory vesicles, are cleaved at Lys-Arg (K-R↓) or Arg-Arg (R-R↓) sequences. Those proteases may require additional auxiliary protein factors for activation, in accordance with their role in regulated secretion only and accordingly are naturally expressed in a highly cell-type specific fashion, usually being expressed only in endocrine cell types. However, the precursors of many growth factors and various plasma proteins that are secreted via unregulated or constitutive pathways have more complex tetrabasic cleavage sites of the general Arg-X-Lys/Arg-Arg (R-X-K/R-R↓) type (Steiner et al., 1992, The new enzymology of precursor processing endoproteases, J. Biol. Chem. 267: 23435-23438; Yanagita et al., 1993, Processing of mutated proinsulin with tetrabasic cleavage sites to mature insulin reflects the expression of furin in non-endocrine cell lines, Endocrinology 133: 639-644; Molloy et al., 1994, Intracellular trafficking and activation of the furin proprotein convertase: localization to the Trans-Golgi network and recycling from the cell surface, EMBO J. 13: 18-33, Vey et al., 1994, Maturation of the trans-Golgi network protease furin: compartmentalization of propeptide removal substrate cleavage, and COOH-terminal truncation, J. Cell. Biol. 122: 1829-1842; Molloy et al., 1992, Human furin is a calcium-dependent serine endoprotease that recognizes the sequences Arg-X-X-Arg and efficiently cleaves anthrax toxin protective antigen, J. Biol. Chem. 267:16396-16402; Rehemtulla et al., 1993, PACE4 is a member of the mammalian propeptidase family that has overlapping but not identical substrate specificity to PACE/Furin, Biochemistry 32:11586-11590). The denotation ‘K/R’ is to be read as ‘K or R’ herein; kex 2 recognizes a dibasic cleavage site and is understood in the present context as a furin family endoprotease belonging to regulated secretion; yeast does not have constitutive secretion per se as do have higher eukaryotic cells, in particular mammalian cells. For the purpose of the present invention, lymphoma proprotein convertase (as described and further referenced in Loo et al., 1997, J. Biological Chemistry, Vol. 272, No. 43, pp. 27116-27123; the tetrabasic cleavage site motif for lymphoma proprotein convertase is further described in there) is understood as being another member of that furin endoprotease family and, having a tetrabasic cleavage site, is belonging to constitutive secretion. In the present context, such endoprotease belonging to constitutive secretion is also termed a ‘constitutive’ endoprotease activity. Further, upon overexpression in CHO cells, lymphoma proprotein convertase was found both to be still Golgi-only localized and not to be shedded to any extend into the supernatant, either. This protease activity is naturally present in lymphoid cell lines such as hybridoma and myeloma cells; myeloma cells truly are plasmacytoma, i.e. B-cell lineage derived cells. Further, non-secreting hybridoma such as SP2/0 or trioma cells may usually encompass such activity.

In the present context, a tetrabasic cleavage site or site motif recognized by the furin family endoproteases is defined as an contiguous tetrapeptide sequence comprising at least three basic residues selected from the group consisting of arginine and lysine. More preferably, the tetrapeptide sequence comprises even four basic residues selected from the group consisting of arginine and lysine. Such cleavage site motif is for instance described in Loo et al., 1997, supra.

According to the present invention, it is also possible that the furin family endoprotease activity is not the activity of a natural occurring furin family endoprotease but that of an artificially created, functional homologue. A functional homologue is defined as a modified furin family enzyme sequence preserving the characteristic features of 1. proteolytic activity in the environment of the late Golgi, 2. enzyme activation taking place after leaving the ER compartment and 3. Golgi-only or late Golgi-only localisation at least in one host cell species, and further having 4. preferably a basic cleavage site, more preferably a tetrabasic cleavage site motif. It is therefore conceivable to employ genetically engineered, in the above sense functional variants of known furin family endoprotease gene products in the present invention. Such variants can be generated e.g. by substitutions, deletions, insertions or truncations of the amino acid and its encoding DNA sequence, respectively. Methods for such are well known in the art and usually comprise specific site directed mutagenesis or generation of diversity by random mutagenesis of which is then followed by selecting desired variants by means of functional assays. Routine methods employed for mutagenesis may be e.g. exposure to alkylating agents or UV irradiation, error-prone PCR or related gene shuffling PCR techniques and are usually performed in microorganisms (Miller, J., Experiments in Molecular Genetics, Cold Spring Harbor Laboratory 1972; Ling et al., 1997, Approaches to DNA Mutagenesis, Analytical biochemistry 254, 157-178; Cadwell et al., 1992, Randomization of genes by PCR mutagenesis in: PCR Methods, Cold Spring Harbor Laboratory Press 1992; Moore et al., 1997, Strategies for the in vitro evolution of protein function, J. Mol. Biol. 272, 336-347). Preferably, a functional homologue according to the above definition is at least 95% homologue at the amino acid level and is at least 70% homologue at the DNA level.

In a further preferred embodiment of the present invention, the furin endoprotease activities are enzyme activities from vertebrate or more preferably mammalian furin endoprotease enzyme. Further strongly preferred, either alone or in conjunction with the immediately preceding embodiment, is that the furin endoprotease activity is a constitutive endoprotease activity belonging to constitutive secretion pathway. Such endoprotease activity has an aforementioned tetrabasic cleavage site characteristically. It must be understood in the present context that antibodies are usually secreted by constitutive secretion; regulated secretion requires sorting to a distinct subset of secretory vesicles. Furin endoproteases functioning in regulated secretion, e.g. insulin processing PC 2 and PC3, will only be active in those particular secretory vesicles; regulated secretion usually results in very large Ca influx. Sorting of protein to those distinct vesicles requires specific sorting signals, at least some of which are protein specific and poorly understood. Further, for the purpose of the present invention, it goes without saying that the level e.g. of endogenous furin/PACE activity may vary. Chinese hamster ovary cells (CHO) have comparatively low endogenous furin activity; a heterologously expressed, furin-cleavable fusion protein may be secreted up to 30-50% by CHO cells in the uncleaved, native form. Overexpression of recombinant, CHO-derived furin enzyme may help to cleave such fusion protein quantitatively. Despite overexpression, careful localization studies demonstrated that all furin activity was still properly Golgi-only localized. However, overexpression of Kex-2 activity in CHO cells, equipped with suitable secretion leader pre-sequence, has been reported to result in widespread expression of Kex-2 in the secretory pathway including the ER; unlike e.g. mammalian proinsulin convertases PC2 and PC3, Kex-2 proved to retain considerable constitutive proteolytic activity under these conditions, resulting in early cleavage in the ER. However, according to the present invention, in case of recombinant host cells only properly Golgi-only or preferably late Golgi-only localized furin family endoprotease activity falls within the scope of the present invention. Localisation signals for proper Golgi-only localisation of active endoprotease, ionic environment and autoproteolytic activation in the ER contributing to the onset of enzymatic activity, are highly species dependent and may fail when protein is heterologously expressed in fairly distant species. Therefore in a further preferred embodiment of the present invention, the host cells according to the present invention are devoid of non-vertebrate, more preferably non-mammalian furin endoprotease activity stemming from native endoprotease enzyme such as e.g. Kex-2 of yeast. What is possible though, is to create properly localized, artificial hybrid enzymes assembled by a localization signal moiety and an active enzyme domain; since approach may not work out simply, a more elaborate combinatorial format for creating functional variants of furin endoproteases having proper Golgi-only or late Golgi-only localization properties should be chosen. Such approach is described for the similar task of glycosylation engineering by means of precisely localized, heterologous glycosyltransferases in Choi et al., Use of combinatorial genetic libraries to humanize N-linked glycosylation in the yeast Pichia pastoris, PNAS 2003, Vol. 100: 5022-5027 and WO 02/00879. WO 02/00879 describes requirements of adequate signal sequences for Golgi localization and pH optima requirements for protease domain activity in detail; said features disclosed therein, in particular the Golgi localization sequences, are herewith incorporated to the present description. In a preferred embodiment of the present invention, suitable functional variants of the active enzymes of the furin endoprotease family according to the present invention do not encompass, i.e. are devoid of such inter-species or chimeric protease enzymes obtained by artificial combination of localization and protease domains of different species origin.—The localization/retention signal sequence of furin is also known: 711-PSDSEEDEG-780 (Takahashi S et al.: J Biol. Chem. 1995 Nov. 24; 270(47):28397-401. There is also a recycling signal to transport furin from the cell membrane back into the cell, probably as a salvage mechanism making up for a minor degree leaky retention in the Golgi, thus preventing surface display or extracellular shedding of furin. For biotechnological application, this is of course an important aspect.

The furin family endoproteases according to the present invention generate proteinaceous fragments with C-terminal basic residues by virtue of their basic cleavage site motif, which basic residues are usually or mostly removed then by carboxypeptidase E, an exopeptidase. A host cell as defined above having suitable carboxypeptidase E activity is a further preferred embodiment of the present invention. ‘Suitable carboxypeptidase E activity’, in accordance with the present invention, is meant to be established in accordance with the present invention if at least 75%, more preferably at least 85%, more preferably at least 95% of protein cleaved by furin endoprotease activity and subsequently secreted is devoid of C-terminal basic (lysine or arginine) residue stemming from endoprotease cleavage site.

Preferably, the fusion polypeptide comprises at least two basic cleavage site motif recognized by a mammalian furin family endoprotease in the way that at least the linker separating the first and second polypeptide is linked to either polypeptide via a cleavage site, more preferably that the at least two cleavage site motifs are further recognized by a constitutively active furin family endoprotease and that the cleavage site motif is a tetrabasic furin family cleavage site motif accordingly. Given the fact that a given, single furin endoprotease may act on slightly varying cleavage site motifs (K/R content) with different kinetics, and that of course multiple different (and constitutive) furin endoprotease activities having different optimal cleavage site motifs and activity levels present in a single cell, it is possible according to the present embodiment that the afore said cleavage site motifs may the same or different.

In a further preferred embodiment, the furin family endoprotease activity is an activity naturally present in the host cell line. That is, no recombinant engineering of furin endoprotease coding genes has taken place in such host cell line but only naturally occurring furin family endoprotease activities are present.

According to the present invention, preferably the host cell is devoid of detectable furin family endoprotease activity that is cleaving the fusion polypeptide in the endoplasmic reticulum, preferably is cleaving the fusion polypeptide at the basic cleavage site motif or motifs of the fusion polypeptide. The above employed wording ‘Golgi-’ or ‘late-Golgi-only’ imply this already. As is nowadays inferable as standard wisdom from cell biology textbooks, the secretory pathway is segmented into at least two very well distinguishable, well-defined compartments, the endoplasmic reticulum where protein folding, core glycosylation and protein assembly along with quality control of protein structure takes place, and the Golgi apparatus. Both morphological/microscopical and biochemical means allow of distinguishing in between these compartments; further, standard subcellular fractionation techniques employing gradient density centrifugation techniques can be employed as is routinely done in the art. Preferably, since the part of the Golgi that is most proximal to the ER, may in certain embodiments of the present invention allow of some recycling of membrane vesicles back to the ER, the furin family endoprotease activity according to the present invention is a late-Golgi only activity. As regards the molecular markers usually employed for distinguishing early (cis), medial and late trans) Golgi, reference is made to the standard textbooks. In the present-context, late-Golgi as a term should be construed as not to exclude the presence of at least some amount of furin endoprotease activity being harbored in secretory vesicles in between the late Golgi and the outer cellular membrane.

In a strongly preferred embodiment according to the present invention, the furin endoprotease activity comprises at least one recombinant furin endoprotease activity. The recombinant activity may be expressed from an episomal or chromosomal expression cassette; it may be heterologous expression of a furin family endoprotease homologue from another species or a functional variant thereof or expression of a host cell furin family endoprotease for achieving an elevated gene dosage. More preferably, such recombinant endoprotease is a constitutive furin family endoprotease activity or a functional variant thereof. Most preferably, such recombinant endoprotease is furin (PACE) endoprotease or lymphoma proprotein convertase (LPC) or a functional variant thereof.

In such way, it is e.g possible to use CHO cells in the present invention for cleaving the fusion polypeptide carrying furin-specific cleavage site motifs quantitative. CHO cells have naturally low level furin endoprotease activity.

In a further preferred embodiment, afore said recombinant such recombinant endoprotease is furin (PACE) endoprotease or lymphoma proprotein convertase (LPC) or a functional variant thereof and the host cell is a CHO host cell.

Suitable media and culture methods for vertebrate and mammalian cell lines are well-known in the art, as described in U.S. Pat. No. 5,633,162 for instance. Examples of standard cell culture media for laboratory flask or low density cell culture and being adapted to the needs of particular cell types are for instance: Roswell Park Memorial Institute (RPMI) 1640 medium (Morre, G., The Journal of the American Medical Association, 199, p. 519 f. 1967), L-15 medium (Leibovitz, A. et al., Amer. J. of Hygiene, 78, 1p.173 ff, 1963), Dulbecco's modified Eagle's medium (DMEM), Eagle's minimal essential medium (MEM), Ham's F12 medium (Ham, R. et al., Proc. Natl. Acad. Sc.53, p288 ff. 1965) or Iscoves' modified DMEM lacking albumin, transferrin and lecithin (Iscoves et al., J. Exp. med. 1, p. 923 ff., 1978). It is known that such culture media can be supplemented with fetal bovine serume (FBS, also called FCS), the latter providing a natural source of a plethora of hormones and growth factors.

For high-density growth of the animal cells in an industrial fed-batch bioreactor according to the present invention, a high-density growth culture medium has to be employed.

According to the present invention, a cell culture medium will be a high-density growth culture medium by definition if the culture medium allows for growth of animal cells up to or in excess of a density of viable cells of 10⁵-10⁶ cells/ml in a conventional fed-batch bioreactor system. Usually, such a medium will comprise 1-10 g/l Glucose or another source of energy, the concentration of glucose being controlled at this level during fed-batch cultivation. Preferably, the medium will comprise at least 2 g/l Glucose, this concentration essentially being controlled during fed-batch fermentation. The medium is isotonic, namely being in the range of 270-320 mOsm/kg, preferably at 280-300 mOsm/kg. Individual preferences of certain cell types, e.g. lymphoid cells, for certain media are well-known in the art, and are complexly correlated with the range, proportion and individual dosing of nutrients. Examples of a high-density growth media suited e.g. for hybridoma cell lines as compared to the standard media mentioned above are given in GB2 251 249 A and EP-435 911 A and e.g. for CHO cells are given in U.S. Pat. No. 5,122,469; EP-435 911 and EP-229 809 do also describe suitable fed-batch regimes for cell culture, as shown in EP-229 809, the fed composition partly making up for elevated nutrient contents of inoculation media used in e.g. EP-435 911; such high-density growth media can be usually supplemented with nutrients such as all amino acids, energy sources such as glucose in the range given above, inorganic salts, vitamins, trace elements (defined as inorganic compounds usually present at final concentrations in the micromolar range), buffers, the four nucleosides or their corresponding nucleotides, antioxidants such as Glutathione (reduced), Vitamin C and other components such as important membrane lipids, e.g. cholesterol or phosphatidylcholine or lipid precursors, e.g. choline or inositol. A high-density medium will be enriched in most or all of these compounds, and will, except for the inorganic salts based on which the osmolarity of the essentially isotonic medium is regulated, comprise them in higher amounts (fortified) than the afore mentioned standard media as can be incurred from GB2251 249 in comparison with RPMI 1640. Preferably, a high-density culture medium according to the present invention is balancedly fortified in that all amino acids except for Tryptophane are in excess of 75 mg/l culture medium. Preferably, in conjunction with the general amino acid requirement, Glutamine and/or Asparagine are jointly in excess of 1 g/l, more preferably of 2 g/l of high-density culture medium. It goes without saying that the latter preferred embodiment is less suitable in case of a recombinant cell line transfected with a Glutamine synthetase (GS) vector (Bebbington et al., 1992, High-level expression of a recombinant antibody from myeloma cells using a glutamine synthetase gene as an amplifiable selectable marker, Bio/Technology 10:169-175; Cockett et al., 1990, High level expression of tissue inhibitor of metalloproteinases in Chinese Hamster Ovary (CHO) cells using Glutamine synthetase gene amplification, Bio/Technology 8: 662-667). In comparison to the dihydrofolate reductase (DHFR) system, the GS system offers a large time advantage during development because highly productive cell lines can often be created from the initial pool of transfectants thus avoiding the need for multiple rounds of selection in the presence of increasing concentrations of selective agent in order to achieve gene amplification (Brown et al., 1992, Process development for the production of recombinant antibodies using the glutamine synthetase (GS) system, Cytotechnology 9:231-236). In such a GS cell line, an excess of e.g. glutamine stemming both from exogenous and endogenous source would lead to production of ammonia which is to be avoided.

It is also possible according to the present invention, to use a cell culture medium that is devoid of fetal calf serum (FCS or FBS), which medium is consequently being termed ‘serum-free’. Cells in serum-free medium generally require insulin and transferrin in a serum-free medium for optimal growth. Transferrin may at least partially be substituted by non-peptide siderophores such as tropolone as described in WO 94/02592. Most cell lines require one or more of synthetic growth factors (comprising recombinant polypeptides), including e.g. epidermal growth factor (EGF), fibroblast growth factor (FGF), insulin like growth factors I and II (IGFI, IGFII), etc. Other classes of factors which may be necessary include: prostaglandins, transport and binding proteins (e.g. ceruloplasmin, high and low density lipoproteins, bovine serum albumin (BSA)), hormones, including steroid-hormones, and fatty acids. Polypeptide factor testing is best done in a stepwise fashion testing new polypeptide factors in the presence of those found to be growth stimulatory. There a several methodological approaches well-known in animal cell culture, an exemplary being described in the following. The initial step is to obtain conditions where the cells will survive and/or grow slowly for 3-6 days after transfer from serum-supplemented culture medium. In most cell types, this is at least in part a function of inoculum density. Once the optimal hormone/growth factor/polypeptide supplement is found, the inoculum density required for survival will decrease. Further, it may also be possible to use modern protein-free media at least with certain host cell lines.

Suitable bioreactors according to the present invention may be any culture system, for instance batch bioreactors such as e.g. airlift bioreactors or stirred bioreactors as routinely employed for high-density animal cell culture. Expediently, for high-density cell culture such bioreactor will be operated in a fed-batch mode. This definition includes continuos feed operation as well. Preferably, fed-batch bioreactors according to the present invention have a volumetric oxygen mass transfer coefficient K_{L}a (as defined in Bailey, J. et al., Biochemical Engineering Fundamentals, McGraw-Hill, N.Y. 1986) of at least 6 h⁻¹, more preferably of at least 10 h⁻¹. Most preferably, a fed-batch bioreactor having said preferred oxygen mass transfer properties according to the present invention is an airlift bioreactor. Airlift bioreactors are well-known to the skilled person and the crucial parameters for reactor design have been well described (for review, see e.g. Chisti, M. et al., 1987, Airlift reactors, Chem. Eng. Commun. 60, 195-242; Koch, A. et al., 1987, Measurement and modeling of mass transport in airlift-loop reactors in relation to the reactor design, Chem. Ing. Tech. 59, 964-965).

In another object of the present invention, the method of the present invention is employed in yeast based on yeast kex-2 activity and the corresponding dibasic cleavage site motif. Kex-2 is the natural furin family endoprotease activity of yeast and does not require any engineering of yeast host cell; it naturally generates active mating factors from prohormone sequences and helps directing them to secretion, whereas constitutive secretion in yeast is targeted to the vacuole. Of course, this requires that naturally occurring, incidental dibasic lysine/arginine motifs within the peptide sequence of the first and second polypeptide are to be avoided, eventually by genetic engineering of the immunoglobuline gene sequence. Further, expression of immunoglobuline usually requires suitable adaptation and engineering of yeast glycosylation, for rendering the resulting product immunologically acceptable for pharmaceutical dosing to humans. Glycosylation engineering in yeast, with the aim of humanized N-glycosylation of expressed, recombinant protein, is described in WO 02/00879 and Choi et al., supra. Since biopharmaceutical antibodies rarely harbor much further glycosylation sites than the conserved Fc-glycosylation site, a minimal biantennary core structure of the complex or oligomannose type of mammals would do. The work of Choi et al. have shown that such down-sizing of N-glycan structures in yeast is not refractory to yeast viability, contrary to general expectations.

An at least biantennary Asn-GlcNAc₄ Man₃ moiety is to be understood as the empirical formula of the minimal N-glycan radical attached to asparagine (Asn). This definition encompasses of course mature, fully sialylated complex N-glycan structures that may be of from bi- up to tetraanntenary and/or may also carry a further bisecting NacGlc residue. For possible constitutions, it is referred to the standard biochemistry textbooks and review articles.

The description of possible and preferred embodiments in the foregoing apply likewise to this and all subsequent objects, where suitable. Namely it is again a further object of the present invention to devise a method for producing an immunoglobuline having Fc receptor activity and/or complement activation activity which immunoglobuline molecule when secreted from a vertebrate host cell comprises at least a first and a second polypeptide chain which two polypeptide chains are different, comprising the steps of firstly, expressing in a vertebrate host cell having Golgi-only or late-Golgi-only resident subtilisin/kexin family endoprotease activity an fusion polypeptide comprising a secretion targeting sequence directing the polypeptide to the secretory pathway and further comprising at least the first and second polypeptide sequences and at least one cleavage site for the said endoprotease activity, secondly having the fusion polypeptide cleaved in the cells by the furin family endoprotease activity into the first and second polypeptide chains and thirdly, harvesting the secreted immunoglobuline.

Precurosor endoproteases of the subtilisin/kexin family (Seidah et al., Ann. N.Y. Acad. Sci. 1999, 885:57-74, The subtilisin/kexin family of precursor convertases. Emphasis on PC1, PC2/7B2, POMC and the novel enzyme SKI-1.) have also been found to be exclusively Golgi localized (Seidah et al., Proc. Natl. Acad. Sci. USA, 1999 96:1321-1326, Mammalian subtilisin/kexin isozyme SKI-1: A widely expressed proprotein convertase with a unique cleavage specificity and cellular localization). Preferably, such kexin family endoprotease is human SKI-1, a mammalian homologue thereof or a functional variant thereof. Further similar to furin family endoproteases, the endoprotease is firstly translated as an inactive proprotein that is proteolytically activated upon exit from the endoplasmic reticulum; further, its activity is similarly Ca2+-dependent. In contrast, SKI-1 and kexin endoproteases in general do not require strongly basic cleavage site motifs but do cleave in case of SKI-1 the sequence RGLT↓SL (arrow indicating the site of cleavage within the cleavage signal sequence motif). This has the advantage of leaving not leaving much basic, terminal residues left (except the Arginine in position 4), since endogenous cellular carboxypeptidase may in some cell types not efficiently remove all basic residues. Terminal basic residues affect pI of protein and are believed to influence plasma half-life of antibody. It is to be recalled that in a preferred embodiment of the above object of the invention, an all-basic terminal tetrabasic cleavage site motif was preferred for reason of allowing removal of basic residues up to the last position 4-basic residue from the carboxyterminus and further for providing good exposure to aqueous solvent and hence efficient cleavage.—By using kexin family proteases, carboxypeptidase trimming is not needed, however, the position -4 arginine is also definitely not to be removed, allowing of more uniform product protein in terms of pI whilst having to take permanent effect of that charged residue into account, too. It should also be mentioned that in contrast e.g. to recombinantly expressed furin, SKI-1 was found to be also located beside the Golgi in endsomal transport vesicles on way to the outer cellular membrane and that further indeed some truncated, soluble version of natural, membrane bound SKI-1 enzyme was found to be steadily shed into the culture medium in 293 cells expressing the natural SKI-1 endoprotease gene (Seidah et al., supra). The reason for naturally occurring, proteolytic truncation of the SKI-1 enzyme in vivo is unknown.

The afore fusion polypeptides comprising the polypeptide sequences of the mature antibody along with the respective furin or kexin family cleavage site motifs, are a further object of the present invention.

### EXAMPLES

### 1. Expression of B72.3 Antibody as a Fusion Polypeptide in CHO Cells and CHO Cells Co-Expressing Recombinant Furin Endoprotease Gene from CHO

The gene structure of mouse-human chimeric antibody cB72.3 is described in Whiffle et al., Protein Eng. 1987, Dec. 1(6):499-505; the original hybridoma cell line B72.3 from which the recombinant chimeric antibody was constructed is obtainable from ATCC as ATCC No.HB-8108. For the purpose of the present experiment, an in-frame fusion is generated by linking the C-terminus of the light chain of cB72.3 via the sequence -Arg-Arg-Lys-Arg-(Gly-Gly-Gly-Gly-Ser)-6-Arg-Arg-Lys-Arg- to the N-terminus of the heavy chain of cB72.3, further eliminating the signal peptide coding sequence from the heavy chain coding sequence's N-terminus.

This in frame fusion is expressed in CHO-K1 cells exactly as described in Yanagita et al., 1993, Endocrinology 133:639-644, except for the fact that the cB72.3 coding sequence in frame-fusion substitutes for the proinsulin in the mammalian expression vector pcDL-SRα296 (Takebe et al., 1988, Mol. Cell. Biol. 8:466-472). The CHO-K1 cells (ATCC CCL-61) are optionally cultured as suspension adapted cells in another medium (Invitrogen CD-CHO or Hyclone HQSFM4), with or without serum-supplementation.

Antibody is harvested from supernatant and tested for Fc-receptor activity by ADCC assay; even in the absence, reproducibly a significant part of the fusion antibody protein expressed is cleaved in the linker portion giving rise to heterodimeric, functional antibody as determined by reducing SDS-PAGE, Western Blotting and staining with light and heavy chain-specific commercial antibodies followed by chemoluminescence detection, allowing semi-quantitative assessment. In CHO cells made stably recombinant with co-expressed furin endoprotease, distinctively more fusion peptide, is cleaved and gives rise to functional antibody.

### 2. Expression of B72.3 Antibody as a Fusion Polypeptide in CHO Cells and 293 Cells in Serum-Free, Protein-Free Cell Culture and Determination of Total Antibody Expression Level

The experiment used essentially the elements and conditions set forth in experiment 1, where not said to be different. In particular, medium-preadapted cells were used; 293 human embryonic kidney cells (HEK293) were from DMSZ (Deutsche Sammlung v. Mikroorganismen und Zellkulturen GmbH, Mascheroder Weg 1b, 38124 Braunschweig, Germany), catalogue number ACC 305.

For expression, transient transfection was employed; a different expression vector, namely the LAP ‘anti-KB-72.3’ vector whose sequence is set forth in table I below, was used for expression this time. A vector chart is given in FIG. 1 of the drawings for this vector. LAP allows of expression of the B72.3 fusion antibody construct, having the linker as described in exp. 1 above; always, apart from negative control, a control transfection with a LAP-72.3 control vector harboring B72.3 devoid of the linker as two separate expression cassettes, in head-to-tail orientation and using exactly the same genetic control elements/promoter, was carried out for obtaining comparative expression level data.

TABLE I
LAP VECTOR 9831 BP DS-DNA CIRCULAR SYN

| DEFINITION | — |
|---|---|
| ACCESSION | — |
| KEYWORDS | — |
| SEGMENT | signal sequence |
| SOURCE | — |
| FEATURES | Location/Qualifiers |
| mRNA | <1 . . . 92 |
| | /note = “CH1” |
| intron | 93 . . . 484 |
| | /note = “In I” |
| primer_bind | complement(334 . . . 353) |
| | /note = “Z0127” |
| primer_bind | 352 . . . 371 |
| | /note = “Y9213” |
| mRNA | 485 . . . 520 |
| | /note = “Hi” |
| intron | 521 . . . 638 |
| | /note = “In II” |
| mRNA | 639 . . . 968 |
| | /note = “CH2” |
| primer_bind | complement(893 . . . 914) |
| | /note = “Y9214” |
| intron | 969 . . . 1065 |
| | /note = “In III” |
| mRNA | 1066 . . . 1388 |
| | /note = “CH3” |
| primer_bind | 1168 . . . 1186 |
| | /note = “Z3494” |
| primer_bind | complement(1367 . . . 1389) |
| | /note = “PCB-HCR1” |
| polyA_signal | 1397 . . . 1639 |
| | /note = “SV40 poly A” |
| primer_bind | complement(1406 . . . 1423) |
| | /note = “1072” |
| protein_bind | 1896 . . . 1901 |
| | /note = “Sal I” |
| misc_feature | 1924 . . . 1924 |
| | /note = “pEE6 ori” |
| rep_origin | 1924 . . . 2512 |
| | /note = “pUC19 ori” |
| misc_RNA | complement(2683 . . . 3543) |
| | /note = “beta lactamase” |
| protein_bind | 3123 . . . 3128 |
| | /note = “Pvu I” |
| misc_signal | 3918 . . . 4261 |
| | /note = “SV40E (and SV40 ori)” |
| mRNA | 4269 . . . 5468 |
| | /note = “GS cDNA” |
| intron | 5471 . . . 6322 |
| | /note = “SV40 intron + poly A” |
| primer_bind | 6326 . . . 6343 |
| | /note = “hCMVInt_HindIII” |
| promoter | 6348 . . . 7490 |
| | /note = “hCMV-MIE promoter” |
| protein_bind | 6384 . . . 6389 |
| | /note = “Mlu I” |
| 5′ UTR | 7491 . . . 7611 |
| | /note = “5′ UT” |
| primer_bind | 7494 . . . 7508 |
| | /note = “D1653” |
| primer_bind | 7533 . . . 7543 |
| | /note = “hCMVEx1_Bsu36I” |
| intron | 7612 . . . 8438 |
| | /note = “intron” |
| primer_bind | 8393 . . . 8416 |
| | /note = “1053” |
| 5′ UTR | 8439 . . . 8455 |
| | /note = “5′ UT” |
| primer_bind | 8455 . . . 8475 |
| | /note = “PCB-VLF1” |
| mRNA | 8471 . . . 8851 |
| | /note = “VL” |
| mRNA | 8471 . . . 8530 |
| | /note = “LC signal seq (cB72.3)” |
| frag | join (8852 . . . >8854, |
| | <9278 . . . >9831) |
| | /note = “298 to 1099 of pCR4-BsiWI- |
| | BstEII frag clock [Split]” |
| frag | join (<8852 . . . >8854, |
| | <9278 . . . >9831) |
| | /note = “1 to 593 of BsiWI VH CH1 |
| | BstEII fragment |
| | [Split]” |
| misc_feature | 9287 . . . 9295 |
| | /note = “QVQ” |
| primer_bind | 9823 . . . >9831 |
| | /note = “BsiWI BstEII 3′ primer” |
| primer_bind | join(<8852 . . . >8854, |
| | <9278 . . . 9305) |
| | /note = “BsiWI BstEII 5′ primer |
| | [Split]” |
| mRNA | 9287 . . . 9628 |
| | /note = “VH” |
| mRNA | 9629 . . . >9831 |
| | /note = “CH1” |
| primer_bind | complement(9769 . . . 9788) |
| | /note = “787” |
| frag | <9278 . . . 9286 |
| | /note = “409 to 435 of pcB72.3 |
| | putative” |
| misc_signal | <9278 . . . 9286 |
| | /note = “Furin cleavage site” |
| frag | 9287 . . . 9301 |
| | /note = “2311 to 2325 of pcB72.3 |
| | copy” |
| frag | <8855 . . . 9277 |
| | /note = “1 to 426 of Untitled8” |
| frag | complement(8855 . . . <9277) |
| | /note = “298 to 723 of pCR4-BsiWI- |
| | XbaI-frag anticlock” |
| frag | complement(>8855 . . . <9277) |
| | /note = “1 to 434 of BsiWI Kappa |
| | Linker XbaI frag [Split]” |
| frag | <8855 . . . 9172 |
| | /note = “11707 to 12027 of pcB72.3 |
| | copy” |
| primer_bind | 9154 . . . 9277 |
| | /note = “BsiWI XbaI 3′ primer” |
| primer_bind | <8855 . . . 8868 |
| | /note = “BsiWI XbaI 5′ primer |
| | [Split]” |
| mRNA | <8855 . . . >9172 |
| | /note = “Kappa [Split]” |
| primer_bind | complement(8867 . . . 8887) |
| | /note = “MRLC3′” |
| primer_bind | complement(9160 . . . >9172) |
| | /note = “PCB-LCR1 [Split]” |
| frag | 9173 . . . >9277 |
| | /note = “322 to 444 of pcB72.3 |
| | putative” |
| misc_structure | 9185 . . . 9274 |
| | /note = “30 aa linker” |
| misc_signal | 9275 . . . >9277 |
| | /note = “Furin cleavage site” |
| frag | 9200 . . . 9214 |
| | /note = “1 to 15 of pcB72.3 copy” |
| frag | 9215 . . . 9229 |
| | /note = “1 to 15 of pcB72.3 copy” |
| frag | 9230 . . . 9244 |
| | /note = “1 to 15 of pcB72.3 copy” |
| frag | 9245 . . . 9259 |
| | /note = “1 to 15 of pcB72.3 copy” |
| frag | 9260 . . . 9274 |
| | /note = “1 to 15 of pcB72.3 copy” |
| frag | <9173 . . . 9184 |
| | /note = “9499 to 9831 of pcB72.3 |
| | copy [Split]” |
| misc_signal | 9173 . . . 9184 |
| | /note = “Furin cleavage site” |
| BASE COUNT | 2447 A 2561 C 2420 G 2403 T 0 OTHER |
| ORIGIN | — |

| | |
|---|---|
| 1 | GGTGACCGTG CCCTCCAGCA CGTTGGGCAC GAAGACCTAC |
| | ACCTGCAACG TAGATCACAA |
| 61 | GCCCAGCAAC ACCAAGGTGG ACAAGAGAGT TGGTGAGAGG |
| | CCAGCACAGG GAGGGAGGGT |
| 121 | GTCTGCTGGA AGCCAGGCTC AGCCCTCCTG CCTGGACGCA |
| | CCCCGGCTGT GCAGCCCCAG |
| 181 | CCCAGGGCAG CAAGGCATGC CCCATCTGTC TCCTCACCCG |
| | GAGGCCTCTG ACCACCCCAC |
| 241 | TCATGCTCAG GGAGAGGGTC TTCTGGATTT TTCCACCAGG |
| | CTCCGGGCAG CCACAGGCTG |
| 301 | GATGCCCCTA CCCCAGGCCC TGCGCATACA GGGGCAGGTG |
| | CTGCGCTCAG ACCTGCCAAG |
| 361 | AGCCATATCC GGGAGGACCC TGCCCCTGAC CTAAGCCCAC |
| | CCCAAAGGCC AAACTCTCCA |
| 421 | CTCCCTCAGC TCAGACACCT TCTCTCCTCC CAGATCTGAG |
| | TAACTCCCAA TCTTCTCTCT |
| 481 | GCAGAGTCCA AATATGGTCC CCCATGCCCA TCATGCCCAG |
| | GTAAGCCAAC CCAGGCCTCG |
| 541 | CCCTCCAGCT CAAGGCGGGA CAGGTGCCCT AGAGTAGCCT |
| | GCATCCAGGG ACAGGCCCCA |
| 601 | GCCGGGTGCT GACGCATCCA CCTCCATCTC TTCCTCAGCA |
| | CCTGAGTTCC TGGGGGGACC |
| 661 | ATCAGTCTTC CTGTTCCCCC CAAAACCCAA GGACACTCTC |
| | ATGATCTCCC GGACCCCTGA |
| 721 | GGTCACGTGC GTGGTGGTGG ACGTGAGCCA GGAAGACCCC |
| | GAGGTCCAGT TCAACTGGTA |
| 781 | CGTGGATGGC GTGGAGGTGC ATAATGCCAA GACAAAGCCG |
| | CGGGAGGAGC AGTTCAACAG |
| 841 | CACGTACCGT GTGGTCAGCG TCCTCACCGT CCTGCACCAG |
| | GACTGGCTGA ACGGCAAGGA |
| 901 | GTACAAGTGC AAGGTCTCCA ACAAAGGCCT CCCGTCCTCC |
| | ATCGAGAAAA CCATCTCCAA |
| 961 | AGCCAAAGGT GGGACCCACG GGGTGCGAGG GCCACATGGA |
| | CAGAGGTCAG CTCGGCCCAC |
| 1021 | CCTCTGCCCT GGGAGTGACC GCTGTGCCAA CCTCTGTCCC |
| | TACAGGGCAG CCCCGAGAGC |
| 1081 | CACAGGTGTA CACCCTGCCC CCATCCCAGG AGGAGATGAC |
| | CAAGAACCAG GTCAGCCTGA |
| 1141 | CCTGCCTGGT CAAAGGCTTC TACCCCAGCG ACATCGCCGT |
| | GGAGTGGGAG AGCAATGGGC |
| 1201 | AGCCGGAGAA CAACTACAAG ACCACGCCTC CCGTGCTGGA |
| | CTCCGACGGC TCCTTCTTCC |
| 1261 | TCTACAGCAG GCTAACCGTG GACAAGAGCA GGTGGCAGGA |
| | GGGGAATGTC TTCTCATGCT |
| 1321 | CCGTGATGCA TGAGGCTCTG CACAACCACT ACACACAGAA |
| | GAGCCTCTCC CTGTCTCTGG |
| 1381 | GTAAATGAGA ATTCATTGAT CATAATCAGC CATACCACAT |
| | TTGTAGAGGT TTTACTTGCT |
| 1441 | TTAAAAAACC TCCCACACCT CCCCCTGAAC CTGAAACATA |
| | AAATGAATGC AATTGTTGTT |
| 1501 | GTTAACTTGT TTATTGCAGC TTATAATGGT TACAAATAAA |
| | GCAATAGCAT CACAAATTTC |
| 1561 | ACAAATAAAG CATTTTTTTC ACTGCATTCT AGTTGTGGTT |
| | TGTCCAAACT CATCAATGTA |
| 1621 | TCTTATCATG TCTGGATCCA TATTCGCGGG CATCACCGGC |
| | GCCACAGGTG CGGTTGCTGG |
| 1681 | CGCCTATATC GCCGACATCA CCGATGGGGA AGATCGGGCT |
| | CGCCACTTCG GGCTCATGAG |
| 1741 | CGCTTGTTTC GGCGTGGGTA TGGTGGCAGG CCCCGTGGCC |
| | GGGGGACTGT TGGGCGCCAT |
| 1801 | CTCCTTGCAT GCACCATTCC TTGCGGCGGC GGTGCTCAAC |
| | GGCCTCAACC TACTACTGGG |
| 1861 | CTGCTTCCTA ATGCAGGAGT CGCATAAGGG AGAGCGTCGA |
| | CCTCGGGCCG CGTTGCTGGC |
| 1921 | GTTTTTCCAT AGGCTCCGCC CCCCTGACGA GCATCACAAA |
| | AATCGACGCT CAAGTCAGAG |
| 1981 | GTGGCGAAAC CCGACAGGAC TATAAAGATA CCAGCCGTTT |
| | CCCCCTGGAA GCTCCCTCGT |
| 2041 | GCGCTCTCCT GTTCCGACCC TGCCGCTTAC CGGATACCTG |
| | TCCGCCTTTC TCCCTTCGGG |
| 2101 | AAGCGTGGCG CTTTCTCATA GCTCACGCTG TAGGTATCTC |
| | AGTTCGGTGT AGGTCGTTCG |
| 2161 | CTCCAAGCTG GGCTGTGTGC ACGAACCCCC CGTTCAGCCC |
| | GACCGCTGCG CCTTATCCGG |
| 2221 | TAACTATCGT CTTGAGTCCA ACCCGGTAAG ACACGACTTA |
| | TCGCCACTGG CAGCAGCCAC |
| 2281 | TGGTAACAGG ATTAGCAGAG CGAGGTATGT AGGCGGTGCT |
| | ACAGAGTTCT TGAAGTGGTG |
| 2341 | GCCTAACTAC GGCTACACTA GAAGAACAGT ATTTGGTATC |
| | TGCGCTCTGC TGAAGCCAGT |
| 2401 | TACCTTCGGA AAAAGAGTTG GTAGCTCTTG ATCCGGCAAA |
| | CAAACCACCG CTGGTAGCGG |
| 2461 | TGGTTTTTTT GTTTGCAAGC AGCAGATTAC GCGCAGAAAA |
| | AAAGGATCTC AAGAAGATCC |
| 2521 | TTTGATCTTT TCTACGGGGT CTGACGCTCA GTGGAACGAA |
| | AACTCACGTT AAGGGATTTT |
| 2581 | GGTCATGAGA TTATCAAAAA GGATCTTCAC CTAGATCCTT |
| | TTAAATTAAA AATGAAGTTT |
| 2641 | TAAATCAATC TAAAGTATAT ATGAGTAAAC TTGGTCTGAC |
| | AGTTACCAAT GCTTAATCAG |
| 2701 | TGAGGCACCT ATCTCAGCGA TCTGTCTATT TCGTTCATCC |
| | ATAGTTGCCT GACTCCCCGT |
| 2761 | CGTGTAGATA ACTACGATAC GGGAGGGCTT ACCATCTGGC |
| | CCCAGTGCTG CAATGATACC |
| 2821 | GCGAGACCCA CGCTCACCGG CTCCAGATTT ATCAGCAATA |
| | AACCAGCCAG CCGGAAGGGC |
| 2881 | CGAGCGCAGA AGTGGTCCTG CAACTTTATC CGCCTCCATC |
| | CAGTCTATTA ATTGTTGCCG |
| 2941 | GGAAGCTAGA GTAAGTAGTT CGCCAGTTAA TAGTTTGCGC |
| | AACGTTGTTG CCATTGCTAC |
| 3001 | AGGCATCGTG GTGTCACGCT CGTCGTTTGG TATGGCTTCA |
| | TTCAGCTCCG GTTCCCAACG |
| 3061 | ATCAAGGCGA GTTACATGAT CCCCCATGTT GTGCAAAAAA |
| | GCGGTTAGCT CCTTCGGTCC |
| 3121 | TCCGATCGTT GTCAGAAGTA AGTTGGCCGC AGTGTTATCA |
| | CTCATGGTTA TGGCAGCACT |
| 3181 | GCATAATTCT CTTACTGTCA TGCCATCCGT AAGATGCTTT |
| | TCTGTGACTG GTGAGTACTC |
| 3241 | AACCAAGTCA TTCTGAGAAT AGTGTATGCG GCGACCGAGT |
| | TGCTCTTGCC CGGCGTCAAT |
| 3301 | ACGGGATAAT ACCGCGCCAC ATAGCAGAAC TTTAAAAGTG |
| | CTCATCATTG GAAAACGTTC |
| 3361 | TTCGGGGCGA AAACTCTCAA GGATCTTACC GCTGTTGAGA |
| | TCCAGTTCGA TGTAACCCAC |
| 3421 | TCGTGCACCC AACTGATCTT CAGCATCTTT TACTTTCACC |
| | AGCGTTTCTG GGTGAGCAAA |
| 3481 | AACAGGAAGG CAAAATGCCG CAAAAAAGGG AATAAGGGCG |
| | ACACGGAAAT GTTGAATACT |
| 3541 | CATACTCTTC CTTTTTCAAT ATTATTGAAG CATTTATCAG |
| | GGTTATTGTC TCATGAGCGG |
| 3601 | ATACATATTT GAATGTATTT AGAAAAATAA ACAAATAGGG |
| | GTTCCGCGCA CATTTCCCCG |
| 3661 | AAAAGTGCCA CCTGACGTCT AAGAAACCAT TATTATCATG |
| | ACATTAACCT ATAAAAATAG |
| 3721 | GCGTATCACG AGGCCCTGAT GGCTCTTTGC GGCACCCATC |
| | GTTCGTAATG TTCCGTGGCA |
| 3781 | CCGAGGACAA CCCTCAAGAG AAAATGTAAT CACACTGGCT |
| | CACCTTCGGG TGGGCCTTTC |
| 3841 | TGCGTTTATA AGGAGACACT TTATGTTTAA GAAGGTTGGT |
| | AAATTCCTTG CGGCTTTGGC |
| 3901 | AGCCAAGCTA GATCCGGCTG TGGAATGTGT GTCAGTTAGG |
| | GTGTGGAAAG TCCCCAGGCT |
| 3961 | CCCCAGCAGG CAGAAGTATG CAAAGCATGC ATCTCAATTA |
| | GTCAGCAACC AGGTGTGGAA |
| 4021 | AGTCCCCAGG CTCCCCAGCA GGCAGAAGTA TGCAAAGCAT |
| | GCATCTCAAT TAGTCAGCAA |
| 4081 | CCATAGTCCC GCCCCTAACT CCGCCCATCC CGCCCCTAAC |
| | TCCGCCCAGT TCCGCCCATT |
| 4141 | CTCCGCCCCA TGGCTGACTA ATTTTTTTTA TTTATGCAGA |
| | GGCCGAGGCC GCCTCGGCCT |
| 4201 | CTGAGCTATT CCAGAAGTAG TGAGGAGGCT TTTTTGGAGG |
| | CCTAGGCTTT TGCAAAAAGC |
| 4261 | TAGCTTGGGG CCACCGCTCA GAGCACCTTC CACCATGGCC |
| | ACCTCAGCAA GTTCCCACTT |
| 4321 | GAACAAAAAC ATCAAGCAAA TGTACTTGTG CCTGCCCCAG |
| | GGTGAGAAAG TCCAAGCCAT |
| 4381 | GTATATCTGG GTTGATGGTA CTGGAGAAGG ACTGCGCTGC |
| | AAAACCCGCA CCCTGGACTG |
| 4441 | TGAGCCCAAG TGTGTAGAAG AGTTACCTGA GTGGAATTTT |
| | GATGGCTCTA GTACCTTTCA |
| 4501 | GTCTGAGGGC TCCAACAGTG ACATGTATCT CAGCCCTGTT |
| | GCCATGTTTC GGGACCCCTT |
| 4561 | CCGCAGAGAT CCCAACAAGC TGGTGTTCTG TGAAGTTTTC |
| | AAGTACAACC GGAAGCCTGC |
| 4621 | AGAGACCAAT TTAAGGCACT CGTGTAAACG GATAATGGAC |
| | ATGGTGAGCA ACCAGCACCC |
| 4681 | CTGGTTTGGA ATGGAACAGG AGTATACTCT GATGGGAACA |
| | GATGGGCACC CTTTTGGTTG |
| 4741 | GCCTTCCAAT GGCTTTCCTG GGCCCCAAGG TCCGTATTAC |
| | TGTGGTGTGG GCGCAGACAA |
| 4801 | AGCCTATGGC AGGGATATCG TGGAGGCTCA CTACCGCGCC |
| | TGCTTGTATG CTGGGGTCAA |
| 4861 | GATTACAGGA ACAAATGCTG AGGTCATGCC TGCCCAGTGG |
| | GAACTCCAAA TAGGACCCTG |
| 4921 | TGAAGGAATC CGCATGGGAG ATCATCTCTG GGTGGCCCGT |
| | TTCATCTTGC ATCGAGTATG |
| 4981 | TGAAGACTTT GGGGTAATAG CAACCTTTGA CCCCAAGCCC |
| | ATTCCTGGGA ACTGGAATGG |
| 5041 | TGCAGGCTGC CATACCAACT TTAGCACCAA GGCCATGCGG |
| | GAGGAGAATG GTCTGAAGCA |
| 5101 | CATCGAGGAG GCCATCGAGA AACTAAGCAA GCGGCACCGG |
| | TACCACATTC GAGCCTACGA |
| 5161 | TCCCAAGGGG GGCCTGGACA ATGCCCGTGG TCTGACTGGG |
| | TTCCACGAAA CGTCCAACAT |
| 5221 | CAACGACTTT TCTGCTGGTG TCGCCAATCG CAGTGCCAGC |
| | ATCCGCATTC CCCGGACTGT |
| 5281 | CGGCCAGGAG AAGAAAGGTT ACTTTGAAGA CCGCGGCCCC |
| | TCTGCCAATT GTGACCCCTT |
| 5341 | TGCAGTGACA GAAGCCATCG TCCGCACATG CCTTCTCAAT |
| | GAGACTGGCG ACGAGCCCTT |
| 5401 | CCAATACAAA AACTAATTAG ACTTTGAGTG ATCTTGAGCC |
| | TTTCCTAGTT CATCCCACCC |
| 5461 | CGCCCCAGAG AGATCTTTGT GAAGGAACCT TACTTCTGTG |
| | GTGTGACATA ATTGGACAAA |
| 5521 | CTACCTACAG AGATTTAAAG CTCTAAGGTA AATATAAAAT |
| | TTTTAAGTGT ATAATGTGTT |
| 5581 | AAACTACTGA TTCTAATTGT TTGTGTATTT TAGATTCCAA |
| | CCTATGGAAC TGATGAATGG |
| 5641 | GAGCAGTGGT GGAATGCCTT TAATGAGGAA AACCTGTTTT |
| | GCTCAGAAGA AATGCCATCT |
| 5701 | AGTGATGATG AGGCTACTGC TGACTCTCAA CATTCTACTC |
| | CTCCAAAAAA GAAGAGAAAG |
| 5761 | GTAGAAGACC CCAAGGACTT TCCTTCAGAA TTGCTAAGTT |
| | TTTTGAGTCA TGCTGTGTTT |
| 5821 | AGTAATAGAA CTCTTGCTTG CTTTGCTATT TACACCACAA |
| | AGGAAAAAGC TGCACTGCTA |
| 5881 | TACAAGAAAA TTATGGAAAA ATATTCTGTA ACCTTTATAA |
| | GTAGGCATAA CAGTTATAAT |
| 5941 | CATAACATAC TGTTTTTTCT TACTCCACAC AGGCATAGAG |
| | TGTCTGCTAT TAATAACTAT |
| 6001 | GCTCAAAAAT TGTGTACCTT TAGCTTTTTA ATTTGTAAAG |
| | GGGTTAATAA GGAATATTTG |
| 6061 | ATGTATAGTG CCTTGACTAG AGATCATAAT CAGCCATACC |
| | ACATTTGTAG AGGTTTTACT |
| 6121 | TGCTTTAAAA AACCTCCCAC ACCTCCCCCT GAACCTGAAA |
| | CATAAAATGA ATGCAATTGT |
| 6181 | TGTTGTTAAC TTGTTTATTG CAGCTTATAA TGGTTACAAA |
| | TAAAGCAATA GCATCACAAA |
| 6241 | TTTCACAAAT AAAGCATTTT TTTCACTGCA TTCTAGTTGT |
| | GGTTTGTCCA AACTCATCAA |
| 6301 | TGTATCTTAT CATGTCTGGA TCTCTAGCTT CGTGTCAAGG |
| | ACGGTGACTG CAGTGAATAA |
| 6361 | TAAAATGTGT GTTTGTCCGA AATACGCGTT TTGAGATTTC |
| | TGTCGCCGAC TAAATTCATG |
| 6421 | TCGCGCGATA GTGGTGTTTA TCGCCGATAG AGATGGCGAT |
| | ATTGGAAAAA TCGATATTTG |
| 6481 | AAAATATGGC ATATTGAAAA TGTCGCCGAT GTGAGTTTCT |
| | GTGTAACTGA TATCGCCATT |
| 6541 | TTTCCAAAAG TGATTTTTGG GCATACGCGA TATCTGGCGA |
| | TAGCGCTTAT ATCGTTTACG |
| 6601 | GGGGATGGCG ATAGACGACT TTGGTGACTT GGGCGATTCT |
| | GTGTGTCGCA AATATCGCAG |
| 6661 | TTTCGATATA GGTGACAGAC GATATGAGGC TATATCGCCG |
| | ATAGAGGCGA CATCAAGCTG |
| 6721 | GCACATGGCC AATGCATATC GATCTATACA TTGAATCAAT |
| | ATTGGCCATT AGCCATATTA |
| 6781 | TTCATTGGTT ATATAGCATA AATCAATATT GGCTATTGGC |
| | CATTGCATAC GTTGTATCCA |
| 6841 | TATCATAATA TGTACATTTA TATTGGCTCA TGTCCAACAT |
| | TACCGCCATG TTGACATTGA |
| 6901 | TTATTGACTA GTTATTAATA GTAATCAATT ACGGGGTCAT |
| | TAGTTCATAG CCCATATATG |
| 6961 | GAGTTCCGCG TTACATAACT TACGGTAAAT GGCCCGCCTG |
| | GCTGACCGCC CAACGACCCC |
| 7021 | CGCCCATTGA CGTCAATAAT GACGTATGTT CCCATAGTAA |
| | CGCCAATAGG GACTTTCCAT |
| 7081 | TGACGTCAAT GGGTGGAGTA TTTACGGTAA ACTGCCCACT |
| | TGGCAGTACA TCAAGTGTAT |
| 7141 | CATATGCCAA GTACGCCCCC TATTGACGTC AATGACGGTA |
| | AATGGCCCGC CTGGCATTAT |
| 7201 | GCCCAGTACA TGACCTTATG GGACTTTCCT ACTTGGCAGT |
| | ACATCTACGT ATTAGTCATC |
| 7261 | GCTATTACCA TGGTGATGCG GTTTTGGCAG TACATCAATG |
| | GGCGTGGATA GCGGTTTGAC |
| 7321 | TCACGGGGAT TTCCAAGTCT CCACCCCATT GACGTCAATG |
| | GGAGTTTGTT TTGGCACCAA |
| 7381 | AATCAACGGG ACTTTCCAAA ATGTCGTAAC AACTCCGCCC |
| | CATTGACGCA AATGGGCGGT |
| 7441 | AGGCGTGTAC GGTGGGAGGT CTATATAAGC AGAGCTCGTT |
| | TAGTGAACCG TCAGATCGCC |
| 7501 | TGGAGACGCC ATCCACGCTG TTTTGACCTC CATAGAAGAC |
| | ACCGGGACCG ATCCAGCCTC |
| 7561 | CGCGGCCGGG AACGGTGCAT TGGAACGCGG ATTCCCCGTG |
| | CCAAGAGTGA CGTAAGTACC |
| 7621 | GCCTATAGAG TCTATAGGCC CACCCCCTTG GCTTCTTATG |
| | CATGCTATAC TGTTTTTGGC |
| 7681 | TTGGGGTCTA TACACCCCCG CTTCCTCATG TTATAGGTGA |
| | TGGTATAGCT TAGCCTATAG |
| 7741 | GTGTGGGTTA TTGACCATTA TTGACCACTC CCCTATTGGT |
| | GACGATACTT TCCATTACTA |
| 7801 | ATCCATAACA TGGCTCTTTG CCACAACTCT CTTTATTGGC |
| | TATATGCCAA TACACTGTCC |
| 7861 | TTCAGAGACT GACACGGACT CTGTATTTTT ACAGGATGGG |
| | GTCTCATTTA TTATTTACAA |
| 7921 | ATTCACATAT ACAACACCAC CGTCCCCAGT GCCCGCAGTT |
| | TTTATTAAAC ATAACGTGGG |
| 7981 | ATCTCCACGC GAATCTCGGG TACGTGTTCC GGACATGGGC |
| | TCTTCTCCGG TAGCGGCGGA |
| 8041 | GCTTCTACAT CCGAGCCCTG CTCCCATGCC TCCAGCGACT |
| | CATGGTCGCT CGGCAGCTCC |
| 8101 | TTGCTCCTAA CAGTGGAGGC CAGACTTAGG CACAGCACGA |
| | TGCCCACCAC CACCAGTGTG |
| 8161 | CCGCACAAGG CCGTGGCGGT AGGGTATGTG TCTGAAAATG |
| | AGCTCGGGGA GCGGGCTTGC |
| 8221 | ACCGCTGACG CATTTGGAAG ACTTAAGGCA GCGGCAGAAG |
| | AAGATGCAGG CAGCTGAGTT |
| 8281 | GTTGTGTTCT GATAAGAGTC AGAGGTAACT CCCGTTGCGG |
| | TGCTGTTAAC GGTGGAGGGC |
| 8341 | AGTGTAGTCT GAGCAGTACT CGTTGCTGCC GCGCGCGCCA |
| | CCAGACATAA TAGCTGACAG |
| 8401 | ACTAACAGAC TGTTCCTTTC CATGGGTCTT TTCTGCAGTC |
| | ACCGTCCTTG ACACGAAGCT |
| 8461 | TGCCGCCACC ATGAGTGTGC CCACTCAGGT CCTGGGGTTG |
| | CTGCTGCTGT GGCTTACAGA |
| 8521 | TGCCAGATGT GACATCCAGA TGACTCAGTC TCCAGCCTCC |
| | CTATCTGTAT CTGTGGGAGA |
| 8581 | AACTGTCACC ATCACATGTC GAGCAAGTGA GAATATTTAC |
| | AGTAATTTAG CATGGTATCA |
| 8641 | ACAGAAACAG GGAAAATCTC CTCAGCTCCT GGTCTATGCT |
| | GCAACAAACT TAGCAGATGG |
| 8701 | TGTGCCATCA AGGTTCAGTG GCAGTGGATC GGGCACACAG |
| | TATTCCCTCA AGATCAACAG |
| 8761 | CCTGCAGTCT GAAGATTTTG GGAGTTATTA CTGCCAACAT |
| | TTTTGGGGTA CTCCGTACAC |
| 8821 | GTTCGGAGGG GGGACCAGGC TGGAAATAAA ACGTACGGTG |
| | GCTGCACCAT CTGTCTTCAT |
| 8881 | CTTCCCGCCA TCTGATGAGC AGTTGAAATC TGGAACTGCC |
| | TCTGTTGTGT GCCTGCTGAA |
| 8941 | TAACTTCTAT CCCAGAGAGG CCAAAGTACA GTGGAAGGTG |
| | GATAACGCCC TCCAATCGGG |
| 9001 | TAACTCCCAG GAGAGTGTCA CAGAGCAGGA CAGCAAGGAC |
| | AGCACCTACA GCCTCAGCAG |
| 9061 | CACCCTGACG CTGAGCAAAG CAGACTACGA GAAACACAAA |
| | GTCTACGCCT GCGAAGTCAC |
| 9121 | CCATCAGGGC CTGAGCTCGC CCGTCACAAA GAGCTTCAAC |
| | AGGGGAGAGT GTAGAAAAAG |
| 9181 | AAGGGGAGGG GGTGGCTCTG GAGGGGGTGG CTCTGGAGGG |
| | GGTGGCTCTG GAGGGGGTGG |
| 9241 | CTCTGGAGGG GGTGGCTCTG GAGGGGGTGG CTCTAGAGAA |
| | AAAAGACAGG TTCAGCTGCA |
| 9301 | GCAGTCTGAC GCTGAGTTGG TGAAACCTGG GGCTTCAGTG |
| | AAGATATCCT GCAAGGCTTC |
| 9361 | TGGCTACACC TTCACTGACC ATGCTATTCA CTGGGCGAAG |
| | CAGAAGCCTG AACAGGGCCT |
| 9421 | GGAATGGATT GGATATATTT CTCCCGGAAA TGATGATATT |
| | AAGTACAATG AGAAGTTCAA |
| 9481 | GGGCAAGGCC ACACTGACTG CAGACAAATC CTCCAGCACT |
| | GCCTACATGC AGCTCAACAG |
| 9541 | CCTGACATCT GAGGATTCTG CAGTGTATTT CTGTAAAAGA |
| | TCGTACTACG GCCACTGGGG |
| 9601 | CCAAGGCACC ACTCTCACAG TCTCCTCAGC CTCCACCAAG |
| | GGCCCATCCG TCTTCCCCCT |
| 9661 | GGCGCCCTGC TCCAGGAGCA CCTCCGAGAG CACAGCCGCC |
| | CTGGGCTGCC TGGTCAAGGA |
| 9721 | CTACTTCCCC GAACCGGTGA CGGTGTCGTG GAACTCAGGC |
| | GCCCTGACCA GCGGCGTGCA |
| 9781 | CACCTTCCCG GCTGTCCTAC AGTCCTCAGG ACTCTACTCC |
| | CTCAGCAGCG T |
| // | |

### Propagation of CHOK1-Suspension Adapted Cells:

Adaptation of cell lines to new, less rich media is described in the scientific literature; an example can further be found in WO 03/064630). CHOK1-‘SV’ cells were routinely propagated in suspension shaker flasks in CD-CHO medium (Invitrogen) supplemented with 6 mM L-glutamine. Seed concentration is 2×10*5 cells/mL, and cells are split every 4 days. Flasks were gassed with 5% CO₂ and incubated at +36.5° C. (between 35.5° C. and 37.0° C.) with orbital shaking at 140 rpm.

### Propagation of HEK293 Cells:

Exactly as per CHOK1SV cells except that the medium is CD-293 (Invitrogen), supplemented with 6 mM L-glutamine.

### Transient Transfection:

Transient transfections were performed using suspension-growing cells. Cells were counted and distributed onto wells of a 24-well plate at 2.5×10⁵ viable cells per well in a DMEM-based medium supplemented with 10% serum and 6 mM L-glutamine, and incubated overnight at +36.5° C. The following day, the conditioned medium was replaced with 1 mL of fresh medium (as above) and the cells incubated for 3 hours at +37° C.

For each transfection, 5 μg of each of the SGVs (HC and LC-SGVs mixed together) or 5 μg of the DGVs were re-suspended in 100 μL transfection medium (OptiMEM, Invitrogen). For positive controls, cells were also transfected with the vector pcB72.3, which encodes heavy chain and light chain genes for an IgG₄/kappa antibody which serves as a model antibody. A negative control (water only) was also included.

For each transfection, 5 μL of Lipofectamine-2000 reagent (Invitrogen) was diluted in 100 μL transfection medium, mixed and left to stand for 5 minute at room temperature. The DNA and diluted Lipofectamine reagent were combined, mixed and further left to stand at ambient temperature for 20 minutes. This 200 μL mixture was then added to a well of the 24-well plate containing the cells, and the cells were incubated for 4 or 10 days at +37° C. The culture supernatant was collected and clarified by centrifugation prior to assay for presence of antibody by assembly ELISA

### Assembly ELISA:

The antibody concentration of samples was determined using a sandwich ELISA which measures assembled human IgG. This involved capture of samples and standard onto a 96 well plate coated with an anti-human Fc antibody. Bound antibody was revealed with an anti-human light chain linked to horseradish peroxidase and the chromogenic substrate TMB. Colour development was controlled to be proportional to the concentration of antibody present in the sample when compared to the standard over the range of measurement.

The Assembly-ELISA titres obtained in this way (arbitrary units) for the samples used in present experiment, detecting both cleaved and uncleaved antibody together, are given in II below:

TABLE II

| | |
|---|---|
| 293-blank | <0.02 |
| 293-anti-KB-72.3 | 6.95 |
| 293-control 72.3 | 0.41 |
| CHOK1sv-blank | <0.02 |
| CHOK1sv-control 72.3 | 17.8 |
| CHOK1sv-anti-KB-72.3 | 20.1 |

Hence, the fusion B72.3 construct was expressed and secreted both in CHO and 293 cells at higher levels than in the heterodimeric B72.3 control construct. In 293 cells, reproducibly, the order of enhancement was huge and completely unexpected. This demonstrates the potential of the method of the present invention. The reasons for this at the molecular level still have to be elucidated.

Aliquots of the samples tested and measured in the Assembly-Elisa were tested for cleavage of the linker moiety and release of heterodimeric protein from the fusion protein by means of both reducing and non-reducing, 5-15% gradient SDS-PAGE, followed by Western blotting (electroblotting at 1 mA/cm² onto Immobulin P membrane (Millipore IPVH00010) and visualization by immunostaining with antibodies specific for the heavy and light chains of the B72.3 antibody: IgG fragments/chains are detected with horseradish peroxidase (HRP) and alkaline phosphatase (AP) conjugated secondary antibodies which are reacted with chromogenic substrate tetramethyl-benzidine (TMB-TMB membrane peroxidase substrate system kit, KPL, Gaithersburg, USA) according to manufacturers manual. For western blotting and immunostaining in general, see Harlow, The antibodies, supra.

Always, neat cleavage of the B72.3 fusion construct was observed, though always presence of still uncleaved fusion antibody was detected, too. Cleavage in 293 cells was observed to take place more efficiently than in CHO cells.

Further details of the experimental methodology are given in the following sections:

The Detection of Fragments of Immunoglobulins by Western Blotting and Probing with Human IgG Specific Antisera in Final Products and In-Process Samples

### 7.13 Antibody Preparations

7.13.1 Primary Antibodies
7.13.1.1 AP (Alkaline Phosphatase) conjugated goat anti-human IgG, Fc fragment specific (Jackson Immunoresearch product number 109-055-098). Store at 5° C.±3° C. for up to 12 months. Use at a dilution of 1 in 1000.
7.13.1.2 HRP conjugated goat anti-human IgG, Fc fragment specific (Jackson Product number 109-035-098). Store at 5° C.±3° C. for up to 12 months. Used at a dilution of 1:2500.
7.13.1.3 AP (Alkaline Phosphatase) conjugated goat anti-Human IgG, Fc (Biogenesis Product number 5211-8104). Store at 5° C.±3° C. for up to 12 months. Use at a dilution of 1 in 1000.
7.13.1.4 HRP conjugated goat anti-human IgG, Fc (Biogenesis Product number 5211-8404). Store at 5° C.±3° C. for up to 12 months. Use at a dilution of 1 in 2000.
7.13.1.5 Sheep anti-human IgG Fd specific, (The Binding Site product number PC075). Store at −20° C.±5° C. until used and then at 5° C.±3° C. for up to 12 months. Use at a dilution of 1 in 1000.
7.13.1.6 HRP conjugated goat anti-human Kappa light chain specific, (Sigma product number A7164). Store at 5° C.±3° C. for up to 12 months. Use at a dilution of 1 in 2000.
7.13.1.7 HRP conjugated goat anti-human IgG (H+L), (Pierce product number 31412). Store at 5° C.±3° C. for up to 12 months. Use at a dilution of 1 in 2000.

7.13.2 Secondary Antibodies
7.13.2.1 AP (Alkaline Phosphatase) conjugate donkey anti-sheep IgG (H+L), (Jackson product number 713-055-147). Store at 5° C.±3° C. for up to 12 months. Use at a dilution of 1 in 5000.
7.13.2.2 Extravidin HRP, (Sigma product number E2886). Store at 5° C.±3° C. for up to 12 months. Use at a dilution of 1 in 5000.

### 7.14 Substrates

7.14.1 TMB substrate kit, (KPL product number 507700). Stored at 5° C.±3° C. for up to 12 months.
4.3 Sulphuric acid is dangerous and can cause burns. Wear gloves and safety glasses when handling. Do not inhale. Use a containment tray. Stand vessel containing water on ice. Add concentrated acid to the water slowly with gentle stirring.
4.4 sodium azide is a skin and respiratory irritant. Avoid skin contact by wearing gloves and handle in a fume hood. It can form explosive compounds with metals. Spillages and waste should be dealt with by following departmental safety procedures.
4.5 All biological materials are potentially hazardous and must be handled with extreme care. Operators should be familiar with and work according to current celltech guidelines as detailed in the safety regulations (consult departmental head).
4.6 Operators should be aware of the potential dangers of tetramethylbenzidine (TMB). Avoid inhalation and skin contact by wearing a face mask and disposable plastic gloves.

### 5.0 Principle

5.1 Standards and samples containing assembled antibody are incubated in wells of microtitre plates pre-coated with goat anti-human IgG. Bound antibody is detected using goat anti-human kappa horseradish peroxidase conjugate. The complex is visualised using the chromogenic substrate tetramethyl benzidine. colour development is proportional to the levels of human IgG in standards or samples.

### 6.0 Responsibilities

6.1 Area Personnel.

### 7.0 Equipment

7.1 Nunc Immunoplates, maxisorp 96 well flat bottomed microtitre plates (product number 4-3945).
7.2 Microplate shaker
7.3 Gilson pipettemans (p20, p200, p1000, p5000) or equivalent plus tips.
7.4 25 ml plastic universals.
7.5 LP3 plastic tubes.
7.6 Multichannel pipettes (Titertek) or equivalent plus tips.
7.7 Plate sealers.
7.8 Plate washer eg. Denley WW004 wellwash.
   Plate reader with 450 nm filter eg. Titertek Multiscan MKII or Biotek EL310.
   Suitable assay processing software eg. Titersoft or Multicalc.

### 8.0 Materials and Reagents

8.1 Distilled/deionised water is used throughout.

8.2 Coating Buffer (0.05M Sodium Carbonate, pH 9.6)
8.2.1

| | |
|---|---|
| Na₂CO₃ | 1.59 g |
| NaHCO₃ | 2.93 g |
| NaN₃ | 0.2 g |

8.2.2 Add distilled water to one litre, and dissolve with stirring.
8.2.3 Store at 4° C. for up to one month.

8.3 Wash Buffer (pH 7.2)
8.3.1

| | | |
|---|---|---|
| NaCl | 58.44 | g |
| Na₂HPO₄ | 11.53 | g |
| NaH₂PO₄•2H₂O | 2.92 | g |
| EDTA | 37.22 | g |
| Tween 20 | 2.0 | ml |
| Butanol | 100.0 | ml |

8.3.2 Add distilled water to 9.9 litres, and dissolve with stirring.
8.3.3 Check pH and adjust to pH7.2 with 1M NaOH if necessary.
8.3.4 Make up to 10 L with distilled water.
8.3.5 Store at ambient temperature for up to one month.

8.4 Blocking Buffer
8.4.1

| | | |
|---|---|---|
| Coating Buffer | 250.0 | ml |
| Casein Hammerstein | 1.25 | g |

8.4.2 Dissolve the casein in the coating buffer with stirring.
8.4.3 Store at 4° C. for up to one month.

8.5 Sample Conjugate Buffer
8.5.1

| | | |
|---|---|---|
| Tris base | 6.05 | g |
| NaCl | 2.92 | g |
| Casein Hammerstein | 1.0 | g |
| Tween 20 | 0.1 | ml |

8.5.2 To Tris base and Nacl add 450 ml distilled water and dissolve with stirring. Adjust pH to 7.0 with concentrated HCl. Add Tween 20 and Casein Hammerstein and make up to 500 ml with distilled water. Store at 4° c. for up to one month.

8.6 Coating Antibody
Fab₂ goat anti-human IgG Fc Jackson Immunoresearch Labs Inc, product number 109-006-098. store in suitable aliquots at 4° C. until manufacturers marked expiry date.

8.7 Anti-Human IgG Kappa Conjugate
Caltag, product number H16007. Aliquot into suitable volumes and store at 4° C. until manufacturers marked expiry date.

8.8 Interassay Control
A sample from a fermentation of a humanised antibody, diluted appropriately to fall on the mid point of the working range of the assay, is stored in 300 μl aliquots (sufficient for a single plate) at −70° C. for up to one year. Absolute value will differ with each standard used, a target value can be obtained by meaning values from 10 assays; A separate control chart should be kept for each standard and value should not vary by 2.5 standard deviations in either direction.

8.9 Standard
Where possible standard material should be the same as the product to be measured. This may involve using a standard provided by another company or Celltech's manufacturing grade product. In either case the quoted concentration will be used to lay down the Elisa standard. If purified material is not available then the standard used should be of the same IgG subclass.

8.10 HRP Substrate
8.10.1 Stock solutions
   8.10.1.1 Tetramethylbenzidine—TMB (ICN Immunobiologicals product number 980502). Dissolve 10 mg TMB in 1 ml dimethylsulphoxide. Store in dark at ambient temperature for up to two weeks.
   8.10.1.2 0.1M sodium acetate/citrate pH 5.0. Dissolve 8.2 g sodium acetate in 900 ml distilled water. Adjust to pH 5.0 with 1M citric acid and make up to a final volume of one litre with distilled water. Store for up to one month at 4° C.
   8.10.1.3 Hydrogen peroxide. Dilute one part 30% H₂O₂ with 300 parts acetate/citrate. Store at ambient temperature for up to two weeks.
8.10.2 Preparation of substrate solution
   This should be carried out immediately prior to use.
   8.10.2.1 Dilute one part TMB stock in 100 parts acetate/citrate buffer.
   8.10.2.2 Add 100 ml H₂O₂ solution to every 10 ml diluted TMB.
   8.10.2.3 Discard after use.

8.11 2.5M H₂SO₄
8.11.1 Add 69.4 ml conc sulphuric acid (sp gr 1.84) to 430.6 ml of distilled water.
   CAUTION: Add acid to water slowly, with stirring, on ice.

### 9.0 Environment

9.1 Laboratory facility unless specified.

### 10.0 Procedure

10.1 Do not allow wells to dry out at any point.
10.2 Plate Coating
   10.2.1 Dilute coating antibody to 1 μg/ml with coating buffer.
   10.2.2 Coat plate with 1 μg/ml of diluted antibody.
   10.2.3 Seal plate and incubate overnight (18 hr±2 hr) at 4° C. on a level surface.
10.3 Wash Plate
   10.3.1 Empty plate and wash×1 in wash buffer.
10.4 Plate Blocking
   10.4.1 Block plate by the addition of 250 μl of blocking buffer to each well and incubate for 1 hour at room temperature.
10.5 Wash Plate
   10.5.1 Empty plate and wash×1 in wash buffer.
10.6 Dilution of standard
   10.6.1 Thaw stock aliquot, allow to reach room temperature and vortex mix.
   10.6.2 Prepare dilutions as follows in assay diluent.
      10.6.2.1 Dilute stock standard to 1000 ng/ml in assay diluent.
      10.6.2.2 Make doubling dilutions by taking a volume of 1000 ng/ml standard and adding an equal volume of diluent, for example 1 ml of standard solution and 1 ml of diluent. Vortex mix and repeat procedure a further 8 times to obtain a range of standard concentrations from 1000 ng/ml to 1.95 ng/ml. (See table below)

| | Vol of Standard | Vol of | |
|---|---|---|---|
| Standard | Solution (μl) | Diluent (μl) | Final Conc. of Standard |
| 1 | | | 1000 ng/ml |
| 2 | 1000 of Standard 1 | 1000 | 500 ng/ml |
| 3 | 1000 of Standard 1 | 1000 | 250 ng/ml |
| 4 | 1000 of Standard 1 | 1000 | 125 ng/ml |
| 5 | 1000 of Standard 2 | 1000 | 62.5 ng/ml |
| 6 | 1000 of Standard 3 | 1000 | 31.3 ng/ml |
| 7 | 1000 of Standard 4 | 1000 | 15.6 ng/ml |
| 8 | 1000 of Standard 5 | 1000 | 7.8 ng/ml |
| 9 | 1000 of Standard 6 | 1000 | 3.9 ng/ml |
| 10 | 1000 of Standard 7 | 1000 | 1.95 ng/ml |

10.7 Dilution of Unknowns
   10.7.1 Dilute as appropriate in assay diluent to give three dilutions in the range 10 to 100 ng/ml.
10.8 Add samples standards and interassay controls to the plate as follows:
   10.8.1 Plate out standards and unknowns into wells (100 μl/well) in duplicate. Add interassay controls in duplicate and add assay diluent to two wells for controls.
   10.8.2 Seal the plate and incubate at room temperature (15-25° C.) for one hour on a plate shaker.
10.9 Wash Plate
   10.9.1 Empty plate and wash×1 in wash buffer.
10.10 Conjugate Addition
   10.10.1 Dilute conjugate as previously determined for relevant batch (see Appendix I) in assay diluent. This has been calculated as 1:20000 for batch No. 0304.
   10.10.2 Add 100 μl to every well.
   10.10.3 seal the plate and incubate at room temperature for one hour on a plate shaker.
10.11 Wash Plate
   10.11.1 Empty plate and wash×2 in wash buffer.
10.12 Substrate
   10.12.1 Make up substrate as in section 8.10.2.
   10.12.2 Add 100 μl to every well.
   10.12.3 Incubate for 10 to 45 minutes at room temperature on a plate shaker. Colour development varies with a number of factors including laboratory temperature and, therefore, incubation time is left to the operator's discretion. For conjugate batch 0304, incubation time is approximately 10 minutes, but this will change for each new conjugate batch used.
   10.12.4 Stop reaction by the addition of 50 μl/well 2.5M H₂SO₄.
   10.12.5 Read absorbances at 450 nm without blanking reader.

### 11.0 Interpretation of Results

11.1 Calculations
11.1.1 Plot A₄₅₀ nm against log concentration of standard to produce a standard curve.
11.1.2 Calculate the amount of assembled antibody in unknown samples from the standard curve.

11.2 Acceptance Criteria
11.2.1 Absorbance value of top standard should be 1.0 absorbance units or above.
11.2.2 Absorbance from assay diluent should be less than 0.1 absorbance unit.
11.2.3 The CV on calibrated data from the working range of the standard curve should not be greater than 15%.
11.2.4 Calibrated values obtained from the IAC should be within 25% of target values, any values falling outside 15% should be highlighted.

### 12.0 Appendicies

12.1 Appendix 1-Calibration of Conjugate Batch

12.2 Appendix 2-Calibration of coating antibody batch

12.3 Appendix 3-Calibration of interassay control

### APPENDIX 1

### Calibration of Conjugate Batch

Titrate out each new conjugate batch across a plate containing 50 ng/ml standard. Also run the current batch of conjugate at its current dilution in a few wells. The conjugate dilution giving equivalent absorbance reading to the current conjugate batch will be used in future ELISA's.

### APPENDIX 2

### Calibration of Coating Antibody Batch

Dilute each new batch of coating antibody (0.5 to 2.0 μg/ml) and coat wells of ELISA plate. Also coat some wells with the current batch to act as a control. Run a standard curve for each coating concentration and the concentration of coating giving equivalent standard absorbance readings to the control antibody will be used in future ELISA's.

### APPENDIX 3

### Calibration of Interassay Control

A sample from late in the fermentation of a cell line producing a humanised antibody, typically containing 10-500 μg/ml of product is chosen to produce a new interassay control (IAC). This sample is titred out in this Elisa and a dilution selected which falls close to the mid point of the standard curve. The bulk fermentation sample is diluted out in sample/conjugate buffer to the selected dilution and aliquoted in 300 μl amounts to provide sufficient IAC samples for 1 years use. These are stored at −70° C. and given a 1 year shelf life.

The new IAC must be measured in the Elisa on ten separate occasions and the results meaned to produce a target value for the new IAC.

1-57. (canceled)

58. Method for producing an immunoglobulin having Fc receptor activity and/or complement activation activity which immunoglobulin molecule when secreted from a vertebrate host cell comprises at least a first and a second polypeptide chain wherein the first polypeptide is an Ig-Light Chain (L) comprising at least a VL and a CL domain and in that the second polypeptide is an Ig-Heavy Chain (H) comprising at least a VH, CH2 and CH3 domain and a hinge domain, comprising the steps of
a. expressing in a vertebrate host cell having Golgi-only or late-Golgi-only resident furin family endoprotease activity a fusion polypeptide comprising a secretion targeting sequence directing the polypeptide to the secretory pathway and further comprising at least the said first and second polypeptide sequences and at least one cleavage site for the said endoprotease activity and wherein the fusion polypeptide comprises the sequences of said first and second polypeptide separated by a linker and
b. having the fusion polypeptide cleaved in the cells by the furin family endoprotease activity into the first and second polypeptide chains and
c. harvesting the secreted immunoglobulin.

59. Method according to claim 58, characterized in that the Ig molecule comprises a CH1 domain.

60. Method according to claim 58, characterized in that the Light and Heavy Chain are separated by a linker, in that the fusion polypeptide comprises at least two cleavage sites for the furin family endoprotease activity and in that the linker is cleaved off from both Heavy and Light Chain by the furin family endoprotease activity by means of said two cleavage sites.

61. Method according to claim 58, characterized in that the furin family endoprotease activity is an activity naturally present in that host cell line.

62. Method according to claim 58, characterized in that the host cell is devoid of furin family endoprotease activity in the endoplasmic reticulum.

63. Method according to claim 58, characterized in that the furin family endoprotease activity is furin endoprotease or lymphoma proprotein convertase or a functional variant thereof.

64. Method according to claim 58, characterized in that the furin family endoprotease activity is a constitutive endoprotease activity.

65. Method according to claim 58, characterized in that the host cell line is a mammalian cell line.

66. Method according to claim 65, characterized in that the host cell has at least one recombinant furin family endoprotease activity which is a homologously expressed mammalian furin family endoprotease naturally present in that host cell line which further is an constitutive furin family endoprotease or furin family endoprotease belonging to constitutive secretion, in this way achieving an elevated expression level of the natural gene product in its native host cell environment.

67. Method according to claim 65, characterized in that the mammalian host cell line are CHO cells.

68. Method according to claim 58, characterized in that the cleavage sites is a contiguous tetrapeptide sequence comprising at least three basic residues selected from the group consisting of arginine and lysine.

69. Method according to claim 68, characterized in that the tetrapeptide sequence comprises four basic residues selected from the group consisting of arginine and lysine.

70. Method according to claim 58, characterized in that the linker is a non-naturally occurring amino acid sequence.

71. Method according to claim 58, characterized in that the linker comprises at least 20 amino acids.

72. Method according to claim 71, characterized in the linker comprises one or several oligomers consisting of only glycine and either serine, threonine or both.

73. Method according to claim 72, characterized in that the linker consists of one or several oligomers consisting of only glycine and either serine, threonine or both.

74. Method according to claim 72, characterized in that the linker comprises at least >60% glycine residues.

75. Method for producing an immunoglobulin having Fc receptor activity and/or complement activation activity which immunoglobulin molecule when secreted from a vertebrate host cell comprises at least a first and a second polypeptide chain wherein the first polypeptide is an Ig-Light Chain (L) comprising at least a VL and a CL domain and in that the second polypeptide is an Ig-Heavy Chain (H) comprising at least a a VH, CH2 and CH3 domain and a hinge domain, comprising the steps of
a. expressing in a vertebrate host cell having Golgi-only or late-Golgi-only resident subtilisin/kexin family endoprotease activity a fusion polypeptide comprising a secretion targeting sequence directing the polypeptide to the secretory pathway and further comprising at least the first and second polypeptide sequences and at least one cleavage site for the said endoprotease activity and wherein the fusion polypeptide comprises the sequences of said first and second polypeptide separated by a linker, and
b. having the fusion polypeptide cleaved in the cells by the subtilisin/kexin family endoprotease activity into the first and second polypeptide chains and
c. harvesting the secreted immunoglobulin.


SEQUENCE LISTING (3 sequences)

SEQ ID NO: 1 - protein, 9 aa, Artificial Sequence
  localization/retention signal
PSDSEEDEG

SEQ ID NO: 2 - protein, 38 aa, Artificial Sequence
  Synthetic construct
RRKRGGGGSGGGGSGGGGSGGGGSGGGGSGGGGSRRKR

SEQ ID NO: 3 - DNA, 9831 bp, Artificial sequence
  Synthetic construct
ggtgaccgtgccctccagcagcttgggcacgaagacctacacctgcaacgtagatcacaagcccagcaacaccaaggtggacaagagagttggtgagaggccagcacagggagggagggtgtctgctggaagccaggctcagccctcctgcctggacgcaccccggctgtgcagccccagcccagggcagcaaggcatgccccatctgtctcctcacccggaggcctctgaccaccccactcatgctcagggagagggtcttctggatttttccaccaggctccgggcagccacaggctggatgcccctaccccaggccctgcgcatacaggggcaggtgctgcgctcagacctgccaagagccatatccgggaggaccctgcccctgacctaagcccaccccaaaggccaaactctccactccctcagctcagacaccttctctcctcccagatctgagtaactcccaatcttctctctgcagagtccaaatatggtcccccatgcccatcatgcccaggtaagccaacccaggcctcgccctccagctcaaggcgggacaggtgccctagagtagcctgcatccagggacaggccccagccgggtgctgacgcatccacctccatctcttcctcagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaaggtgggacccacggggtgcgagggccacatggacagaggtcagctcggcccaccctctgccctgggagtgaccgctgtgccaacctctgtccctacagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaatgagaattcattgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccatattcgcgggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgacctcgggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctgatggctctttgcggcacccatcgttcgtaatgttccgtggcaccgaggacaaccctcaagagaaaatgtaatcacactggctcaccttcgggtgggcctttctgcgtttataaggagacactttatgtttaagaaggttggtaaattccttgcggctttggcagccaagctagatccggctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctagcttggggccaccgctcagagcaccttccaccatggccacctcagcaagttcccacttgaacaaaaacatcaagcaaatgtacttgtgcctgccccagggtgagaaagtccaagccatgtatatctgggttgatggtactggagaaggactgcgctgcaaaacccgcaccctggactgtgagcccaagtgtgtagaagagttacctgagtggaattttgatggctctagtacctttcagtctgagggctccaacagtgacatgtatctcagccctgttgccatgtttcgggaccccttccgcagagatcccaacaagctggtgttctgtgaagttttcaagtacaaccggaagcctgcagagaccaatttaaggcactcgtgtaaacggataatggacatggtgagcaaccagcacccctggtttggaatggaacaggagtatactctgatgggaacagatgggcacccttttggttggccttccaatggctttcctgggccccaaggtccgtattactgtggtgtgggcgcagacaaagcctatggcagggatatcgtggaggctcactaccgcgcctgcttgtatgctggggtcaagattacaggaacaaatgctgaggtcatgcctgcccagtgggaactccaaataggaccctgtgaaggaatccgcatgggagatcatctctgggtggcccgtttcatcttgcatcgagtatgtgaagactttggggtaatagcaacctttgaccccaagcccattcctgggaactggaatggtgcaggctgccataccaactttagcaccaaggccatgcgggaggagaatggtctgaagcacatcgaggaggccatcgagaaactaagcaagcggcaccggtaccacattcgagcctacgatcccaaggggggcctggacaatgcccgtggtctgactgggttccacgaaacgtccaacatcaacgacttttctgctggtgtcgccaatcgcagtgccagcatccgcattccccggactgtcggccaggagaagaaaggttactttgaagaccgcggcccctctgccaattgtgacccctttgcagtgacagaagccatcgtccgcacatgccttctcaatgagactggcgacgagcccttccaatacaaaaactaattagactttgagtgatcttgagcctttcctagttcatcccaccccgccccagagagatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctctagcttcgtgtcaaggacggtgactgcagtgaataataaaatgtgtgtttgtccgaaatacgcgttttgagatttctgtcgccgactaaattcatgtcgcgcgatagtggtgtttatcgccgatagagatggcgatattggaaaaatcgatatttgaaaatatggcatattgaaaatgtcgccgatgtgagtttctgtgtaactgatatcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacatggctctttgccacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccacgcgaatctcgggtacgtgttccggacatgggctcttctccggtagcggcggagcttctacatccgagccctgctcccatgcctccagcgactcatggtcgctcggcagctccttgctcctaacagtggaggccagacttaggcacagcacgatgcccaccaccaccagtgtgccgcacaaggccgtggcggtagggtatgtgtctgaaaatgagctcggggagcgggcttgcaccgctgacgcatttggaagacttaaggcagcggcagaagaagatgcaggcagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttgacacgaagcttgccgccaccatgagtgtgcccactcaggtcctggggttgctgctgctgtggcttacagatgccagatgtgacatccagatgactcagtctccagcctccctatctgtatctgtgggagaaactgtcaccatcacatgtcgagcaagtgagaatatttacagtaatttagcatggtatcaacagaaacagggaaaatctcctcagctcctggtctatgctgcaacaaacttagcagatggtgtgccatcaaggttcagtggcagtggatcgggcacacagtattccctcaagatcaacagcctgcagtctgaagattttgggagttattactgccaacatttttggggtactccgtacacgttcggaggggggaccaggctggaaataaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtagaaaaagaaggggagggggtggctctggagggggtggctctggagggggtggctctggagggggtggctctggagggggtggctctggagggggtggctctagagaaaaaagacaggttcagctgcagcagtctgacgctgagttggtgaaacctggggcttcagtgaagatatcctgcaaggcttctggctacaccttcactgaccatgctattcactgggcgaagcagaagcctgaacagggcctggaatggattggatatatttctcccggaaatgatgatattaagtacaatgagaagttcaagggcaaggccacactgactgcagacaaatcctccagcactgcctacatgcagctcaacagcctgacatctgaggattctgcagtgtatttctgtaaaagatcgtactacggccactggggccaaggcaccactctcacagtctcctcagcctccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgt